(19) United States
(12) Patent Application Publication
(10) Pub. No.: US 2026/0337236 A1
(43) Pub. Date: Oct. 29, 2026

(54) PROCESS FOR THE PREPARATION AND TERMINAL MODIFICATION OF A SEED-DERIVED ELEVEN-RESIDUE PEPTIDE FROM PHOENIX DACTYLIFERA

(71) Applicants: Saad Ahmad H. ALMALKI, Jeddah (SA); Karem IBRAHEM, Jeddah (SA); Bandar Hasan SALEH, Jeddah (SA); Abdelbagi ALFADIL, Jeddah (SA); Hatoon A. NIYAZI, Jeddah (SA); Hanouf A. NIYAZI, Jeddah (SA); Mona A ALQARNI, Jeddah (SA); Ohood S ALHARBI, Rabigh (SA); Nojoud FAQERAH, Rabigh (SA); Turki ALHOMRA, Rabigh (SA); Ahmed A. KHAN, Makkah (SA); Ehssan MOGLAD, Alkharj (SA); Thamir M. EID, Jeddah (SA); Azizah Salim BAWADOOD, Al-Kharj (SA); Mustafa ZEYADI, Jeddah (SA); Fahad AL-ABBASI, Jeddah (SA); Hisham N. ALTAYB, Jeddah (SA)

(72) Inventors: Saad Ahmad H. ALMALKI, Jeddah (SA); Karem IBRAHEM, Jeddah (SA); Bandar Hasan SALEH, Jeddah (SA); Abdelbagi ALFADIL, Jeddah (SA); Hatoon A. NIYAZI, Jeddah (SA); Hanouf A. NIYAZI, Jeddah (SA); Mona A ALQARNI, Jeddah (SA); Ohood S ALHARBI, Rabigh (SA); Nojoud FAQERAH, Rabigh (SA); Turki ALHOMRA, Rabigh (SA); Ahmed A. KHAN, Makkah (SA); Ehssan MOGLAD, Alkharj (SA); Thamir M. EID, Jeddah (SA); Azizah Salim BAWADOOD, Al-Kharj (SA); Mustafa ZEYADI, Jeddah (SA); Fahad AL-ABBASI, Jeddah (SA); Hisham N. ALTAYB, Jeddah (SA)

(21) Appl. No.: 19/395,781

(22) Filed: Nov. 20, 2025

(51) Int. Cl. C07K 14/415

(52) U.S. Cl. CPC C07K 14/415

(57) ABSTRACT

The present disclosure relates to process for the preparation and terminal modification of a seed-derived eleven-residue peptide from phoenix dactylifera. In more particular manner, the present invention aims to evaluate the synergistic antibacterial activity of thymoquinone (TQ), a major bioactive component of Nigella sativa, in combination with two chemically modified antimicrobial peptides (AMPs), H3 and H5, derived from Phoenix dactylifera (date palm), against MRSA clinical isolates. The synergistic interactions between thymoquinone and the modified peptides are assessed using checkerboard assays and time-kill kinetics. The mechanisms underlying observed synergistic effects are elucidated through membrane permeability assessments, and molecular docking studies. Thymoquinone exhibited bactericidal activity at a concentration of 64 μg/mL. The peptides H3 and H5 showed inhibitory activity at 256 μg/mL. When TQ was combined with peptide H3, the MIC of TQ was reduced to 32 μg/mL, indicating a significant synergistic effect.

### FIELD OF THE INVENTION

The present disclosure relates to Process for the Preparation and Terminal Modification of a Seed-Derived Eleven-Residue Peptide from Phoenix dactylifera. In more particular manner, the present invention aims to evaluate the synergistic antibacterial activity of thymoquinone (TQ), a major bioactive component of Nigella sativa, in combination with two chemically modified antimicrobial peptides (AMPs), H3 and H5, derived from Phoenix dactylifera (date palm), against MRSA clinical isolates.

### BACKGROUND OF THE INVENTION

Antimicrobial resistance (AMR) represents one of the most significant global health challenges of the 21st century. The World Health Organization has identified AMR as a critical threat that could potentially lead to 10 million deaths annually by 2050 if left unaddressed. Among the various resistant pathogens, methicillin-resistant Staphylococcus aureus (MRSA) remains particularly concerning due to its prevalence in both healthcare and community settings. MRSA infections are associated with increased morbidity, mortality, and healthcare costs compared to infections caused by susceptible strains.

The emergence and spread of MRSA have been driven by the selective pressure exerted by widespread antibiotic use, coupled with the remarkable genetic plasticity of S. aureus that facilitates rapid adaptation to antimicrobial challenges. Conventional treatment options for MRSA infections have become increasingly limited, with vancomycin often serving as the last resort. However, the emergence of vancomycin-intermediate and vancomycin-resistant S. aureus strains has further complicated the therapeutic landscape, underscoring the urgent need for novel antimicrobial strategies.

Natural products have historically been a rich source of antimicrobial compounds, with approximately 60% of approved antibiotics derived from natural sources. The exploration of plant-derived compounds and their derivatives represents a promising approach to address the challenge of antimicrobial resistance. Two such promising candidates are thymoquinone, derived from Nigella sativa (black seed), and antimicrobial peptides isolated from date palm (Phoenix dactylifera).

Thymoquinone (2-isopropyl-5-methyl-1,4-benzoquinone) is the principal bioactive constituent of Nigella sativa essential oil, comprising 30-48% of its volatile oil. This compound has garnered significant scientific interest due to its diverse pharmacological properties, including anti-inflammatory, antioxidant, anticancer, and antimicrobial activities. The antimicrobial activity of thymoquinone against various pathogens, including S. aureus, has been documented in several studies. Thymoquinone exhibits significant antibacterial activity against both methicillin-susceptible and methicillin-resistant S. aureus strains, with minimum inhibitory concentrations (MICs) ranging from 8 to 64 μg/mL. The proposed mechanisms of action include disruption of bacterial cell membrane integrity, inhibition of biofilm formation, and interference with bacterial quorum sensing systems.

Despite its promising antimicrobial properties, the clinical application of thymoquinone faces several challenges, including limited water solubility, poor bioavailability, and rapid elimination. These limitations have prompted research into combination therapies and chemical modifications to enhance its therapeutic potential.

Date palm (Phoenix dactylifera) has been cultivated for thousands of years in arid regions and holds significant cultural and economic importance in many countries. Beyond its nutritional value, various parts of the date palm have been used in traditional medicine for the treatment of diverse ailments, including infectious diseases. Recent scientific investigations have revealed that date palm seeds contain bioactive peptides with antimicrobial properties. These peptides typically consist of 10-50 amino acid residues and possess amphipathic structures that facilitate their interaction with bacterial membranes. The antimicrobial activity of these peptides is attributed to their ability to disrupt bacterial membrane integrity, leading to cell lysis and death.

Chemical modifications of naturally occurring peptides, such as acetylation, amidation, or cyclization, can enhance their antimicrobial efficacy, stability, and selectivity. Specifically, the acetylation of lysine residues in antimicrobial peptides has been shown to increase their hydrophobicity, thereby improving their interaction with bacterial membranes while potentially reducing their susceptibility to proteolytic degradation.

Combination therapy, involving the concurrent use of multiple antimicrobial agents, represents a promising strategy to combat resistant pathogens. Synergistic interactions, where the combined effect of two agents exceeds the sum of their individual effects, can lead to enhanced antimicrobial efficacy, reduced dosage requirements, and potentially delayed resistance development. The concept of synergy is particularly relevant in the context of natural products, which often contain multiple bioactive compounds that may interact favorably. The combination of compounds with distinct mechanisms of action can target multiple bacterial components simultaneously, making it more challenging for bacteria to develop resistance.

Previous studies have demonstrated synergistic interactions between various natural products and conventional antibiotics against MRSA. For instance, synergy between plant-derived phenolic compounds and aminoglycosides has been reported, while synergistic effects between antimicrobial peptides and conventional antibiotics have been observed.

However, there remains a need for novel antimicrobial strategies that can effectively combat MRSA and other resistant pathogens through synergistic combinations of natural antimicrobial agents. Therefore the present invention aims to evaluate the potential synergistic antimicrobial effects of thymoquinone and chemically modified peptides derived from date palm seeds against methicillin-resistant Staphylococcus aureus. The present invention aims to address teh challenge of antimicrobial resistance in MRSA infections.

### SUMMARY OF THE INVENTION

The present disclosure relates to a method to evaluate the synergistic antibacterial activity of thymoquinone (TQ), a major bioactive component of Nigella sativa, in combination with two chemically modified antimicrobial peptides (AMPs), H3 and H5, derived from Phoenix dactylifera (date palm), against MRSA clinical isolates. For said evaluation, minimum inhibitory concentrations (MICs) are determined using broth microdilution techniques. Combination therapy showed enhanced bactericidal effects compared to monotherapy, suggesting improved membrane disruption and increased bacterial susceptibility. The findings highlight the potential of combining chemically modified plant peptides with phytochemicals like thymoquinone to develop new antimicrobial strategies against resistant pathogens.

An object of the present disclosure is to provide a method to evaluate the synergistic antimicrobial effects of thymoquinone and chemically modified peptides against methicillin-resistant Staphylococcus aureus.

Another object of the present disclosure is to assess the potential synergistic interactions between thymoquinone and the modified peptides using checkerboard assays and time-kill kinetics.

Another object of the present disclosure is to elucidate the mechanisms underlying any observed synergistic effects through membrane permeability assessments, and molecular docking studies.

Yet, another object of the present disclosure is to address the problem of MRSA and other resistant pathogens, by combining the chemically modified peptide from date palm and thymoquinone.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a table showing the synthesized chemically modified peptides, in accordance with an embodiment of the present disclosure;

FIG. 2(a) illustrates a table showing Non-bonded Interaction Summary Between the peptide (NCSIHGDIPAY) and PBP2a, in accordance with an embodiment of the present disclosure;

FIG. 2(b) illustrates a table showing Non-bonded Interaction Summary Between the peptide (NCSIHGDIPAY) and PBP2a, in accordance with an embodiment of the present disclosure;

FIG. 2(c) illustrates a table showing Non-bonded Interaction Summary Between the peptide (NCSIHGDIPAY) and PBP2a, in accordance with an embodiment of the present disclosure;

FIG. 3 illustrates a 3D interaction between peptides atoms and PBP2a protein amino acids, in accordance with an embodiment of the present disclosure;

FIG. 4 illustrates a detailed interaction between peptides atoms and protein amino acids, in accordance with an embodiment of the present disclosure; and

FIG. 5 illustrates a flow chart for a process for the preparation of a chemically-modified peptide derived from seed material.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

The present invention relates to a method for combining thymoquinone, a major bioactive component of Nigella sativa, in combination with two chemically modified antimicrobial peptides (AMPs), H3 and H5, derived from Phoenix dactylifera (date palm), in order to provide synergestic antimicrobial effects against methicillin-resistant Staphylococcus aureus. In order to assess the potential synergistic interactions between thymoquinone and the modified peptides, checkerboard assays and time-kill kinetics are performed. Additionally, membrane permeability assessment and molecular docking studies are performed to elucidate the mechanism underlying any observed synergistic effects. Furthermore, Minimum inhibitory concentrations (MICs) were determined using broth microdilution techniques.

For in silico analysis, chemically modified peptides are derived from the Phoenix dactylifera plant, wherein the ture lectin protein, specially the LRR receptor-like serine/threonine-protein kinase of Phoenix dactylifera, was selected. For the screening of putative peptides, the protein sequence is analyzed to identify potential peptide fragments, using a peptide cutter tool. Enzymatic digestion was simulated using three gastric proteases, pepsin (pH 1.3), trypsin, and chymotrypsin, under default settings. Peptides ranging between 10 and 50 amino acids in length were selected. The peptide sequence NCSIHGDIPAY, which achieved the highest docking score, was chemically modified through three different strategies: C-terminal amidation combined with N-terminal acetylation; N-terminal benzoyl group modification; and N-terminal FITC-PEG2 conjugation. Each of these modified peptides was synthesized commercially with a purity of at least 98%, incorporating distinct terminal alterations. The peptides, consisting of 11 amino acids, were produced in amounts ranging from 20 to 24 mg. These structural modifications highlight their promising potential for antibacterial applications. Three candidate polypeptides were identified from Phoenix dactylifera, labeled as U366M194G0-1, U366M194G0-3, and U366M194G0-5. For prediction of peptide activity and toxicity, the peptide calculator is utilized to evaluate the bacterial activity, hydrophobicity, hydrophilicity, and net charge of the identified peptides, aiding in the prediction of their cell-penetrating capabilities and other physicochemical properties. Additionally, ToxinPred was employed to assess the toxicity potential of the peptides, distinguishing between toxic and non-toxic candidates. The molecular docking analysis is carried out, wherein an essential protein from Methicillin-Resistant Staphylococcus aureus (PBP2a), known for its role in beta-lactam antibiotic resistance, was selected for computational analysis. The crystal structure of PBP2a was retrieved from the Protein Data Bank (PDB ID: 6h5o). Following protein preparation for docking, the active site was identified. Docking between bacterial proteins and peptide was carried out using the HPEPDOCK server and the resulting interactions were visualized.

In an embodiment, synergetic effect of chemically modified peptide from date palm and thymoquinone is investigated against methicillin-resistant Staphylococcus aureus (MRSA). The antimicrobial properties of date palm peptides and thymoquinone, is systematically evaluated, individually, followed by an assessment of their combined effect against MRSA strains. The experimental design incorporates multiple complementary techniques to ensure robust and reliable results, enabling a thorough understanding of the synergistic mechanisms underlying the observed antimicrobial effects.

The methicillin-resistant Staphylococcus aureus (MRSA) strain is utilized in this invention, wherein the obtained strain is maintained on Mueller-Hinton agar plates, and stored as glycerol stocks at −80 °C. Prior to each experiment, bacterial cultures were revived by streaking onto fresh Mueller-Hinton agar plates and incubating at 37 °C for 24 hours. For experimental procedures, single colonies were inoculated into Mueller-Hinton broth and incubated overnight at 37 °C.

Thymoquinone (2-isopropyl-5-methyl-1,4-benzoquinone, TQ) is obtained, having a purity exceeding 98% as confirmed by high-performance liquid chromatography (HPLC). Due to its hydrophobic nature, TQ is initially dissolved in dimethyl sulfoxide (DMSO) to prepare a stock solution of 10 mg/mL. The final concentration of DMSO in all experimental wells is maintained below 1% to avoid solvent interference with antimicrobial activity. The stock solution is filter-sterilized using a 0.22 μm membrane filter, aliquoted, and stored at −20 °C protected from light to prevent oxidative degradation. Fresh dilutions are prepared in Mueller-Hinton broth immediately before each experiment to ensure stability and consistency of the compound.

The peptides H3 and H5 were derived from date palm (Phoenix dactylifera) seeds through a multi-step extraction and purification process. Initially, date palm seeds were collected from mature fruits, thoroughly washed with distilled water, air-dried, and ground into fine powder using a laboratory mill. The powder was defatted using n-hexane in a Soxhlet apparatus for 8 hours, followed by air-drying to remove residual solvent. Protein extraction was performed by suspending the defatted powder in phosphate-buffered saline (PBS, pH 7.4) at a ratio of 1:10 (w/v) and stirring continuously for 4 hours at 4 °C. The mixture was centrifuged at 10,000× g for 30 minutes at 4 °C, and the supernatant was collected. Ammonium sulfate precipitation was performed at 80% saturation, followed by centrifugation at 12,000×g for 45 minutes. The precipitate was resuspended in a minimal volume of PBS and dialyzed against the same buffer using a 3.5 kDa molecular weight cut-off membrane for 48 hours with regular buffer changes. The dialyzed protein extract was subjected to size-exclusion chromatography using a Sephadex G-50 column equilibrated with PBS. Fractions were collected and analyzed for protein content using the Bradford assay. Promising fractions were pooled and further purified using reverse-phase high-performance liquid chromatography (RP-HPLC) on a C18 column with a linear gradient of acetonitrile (0-60%) containing 0.1% trifluoroacetic acid. The eluted peptides were monitored at 220 and 280 nm. The purified peptides were characterized by matrix-assisted laser desorption/ionization time-of-flight mass spectrometry (MALDI-TOF MS) and N-terminal sequencing using Edman degradation. Based on these analyses, two peptides, designated H3 and H5, were selected for further study due to their potential antimicrobial properties. Chemical modification of the peptides involved selective acetylation of lysine residues to enhance their hydrophobicity and potentially improve their interaction with bacterial membranes. The acetylation was performed by treating the peptides with acetic anhydride at a molar ratio of 1:50 (peptide: acetic anhydride) in 50 mM ammonium bicarbonate buffer (pH 8.0) for 1 hour at room temperature. The reaction was quenched by adding an equal volume of 1 M Tris-HCl (pH 8.0). The modified peptides were purified by RP-HPLC, and the extent of acetylation was confirmed by MALDI-TOF MS. The purified modified peptides were lyophilized and stored at −20 °C until further use. For experimental purposes, stock solutions of peptides H3 and H5 were prepared at a concentration of 10 mg/mL in sterile distilled water, filter-sterilized, aliquoted, and stored at −20 °C. Working solutions were freshly prepared in Mueller-Hinton broth before each experiment.

In an embodiment, various equipment are utilized for evaluating the effects of synergetic effect of chemically modified peptide from date palm and thymoquinone against methicillin-resistant Staphylococcus aureus (MRSA), including: a spectrophotometer (Thermo Scientific, Waltham, MA, USA), microplate reader (BioTek, Winooski, VT, USA), HPLC system (Waters, Milford, MA, USA), MALDI-TOF mass spectrometer (Bruker Daltonics, Billerica, MA, USA), protein sequencer (Applied Biosystems, Foster City, CA, USA), scanning electron microscope (JEOL, Tokyo, Japan), flow cytometer (BD Biosciences, San Jose, CA, USA), and equipment such as centrifuges, incubators, and biosafety cabinets.

The minimum inhibitory concentrations (MICs) of thymoquinone and peptides H3 and H5 against MRSA were determined using the broth microdilution technique with slight modification than standard technique, wherein Two-fold serial dilutions of thymoquinone (ranging from 2 to 1024 μg/mL) and peptides H3 and H5 (ranging from 2 to 1024 μg/mL) were prepared in Mueller-Hinton broth in 96-well microtiter plates. Each well contained 100 μL of the antimicrobial agent at the appropriate concentration. Bacterial suspensions were prepared from overnight cultures and adjusted to 0.5 McFarland standard, then diluted 1:150 in Mueller-Hinton broth to achieve a final inoculum of approximately 5×10⁵ CFU/mL. A 100 μL aliquot of this suspension was added to each well, resulting in a final volume of 200 μL per well. Positive control wells contained bacterial suspension without antimicrobial agents, while negative control wells contained only Mueller-Hinton broth. Additionally, vehicle control wells containing DMSO at the highest concentration used in the test wells were included to ensure that the solvent did not affect bacterial growth. The plates were incubated at 37 °C for 24 hours, after which the MIC was determined as the lowest concentration of the antimicrobial agent that completely inhibited visible bacterial growth. To confirm the results, 20 μL of 0.01% resazurin solution was added to each well, and the plates were incubated for an additional 2 hours. A color change from blue to pink indicated viable bacteria, while wells remaining blue confirm the complete growth inhibition. Each experiment was performed in triplicate on three separate occasions to ensure reproducibility. The median value was reported as the MIC for each antimicrobial agent.

The interaction between thymoquinone and peptides H3 and H5 was evaluated using the checkerboard assay is used for assessing antimicrobial synergy, wherein thymoquinone was serially diluted along the x-axis of a 96-well microtiter plate (ranging from 0 to 64 μg/mL), while each peptide was serially diluted along the y-axis (ranging from 0 to 256 μg/mL). This created a matrix of combinations with varying concentrations of both compounds. Each well contained 50 μL of thymoquinone at the appropriate concentration, 50 μL of peptide (either H3 or H5) at the appropriate concentration, and 100 μL of bacterial suspension (5×10\hat{ }5 CFU/mL). Control wells included bacterial growth controls (no antimicrobial agents), sterility controls (no bacteria), and individual antimicrobial agent controls. The plates were incubated at 37 °C for 24 hours, after which growth inhibition was assessed visually and confirmed using resazurin. The fractional inhibitory concentration index (FICI) was calculated to quantify the interaction between thymoquinone and each peptide using the following formula:

$$FICI = ( {MIC}_{A}^{combi} \div {MIC}_{A}^{alone} ) + ( {MIC}_{B}^{combi} \div {MIC}_{B}^{alone} )$$

The interaction was interpreted as synergistic if FICI≤0.5, additive if 0.5<FICI≤1, indifferent if 1<FICI<4, and antagonistic if FICI≥4.

Time-kill kinetics assays were performed to evaluate the bactericidal activity of thymoquinone and peptides H3 and H5, both individually and in combination, over time. MRSA suspensions (approximately 5×10⁵ CFU/mL) were exposed to the following treatments in Mueller-Hinton broth: (1) thymoquinone alone at 0.5×MIC, 1×MIC, and 2×MIC; (2) peptide H3 alone at 0.5×MIC, 1×MIC, and 2×MIC; (3) peptide H5 alone at 0.5×MIC, 1×MIC, and 2×MIC; (4) thymoquinone+peptide H3 at various combinations of their respective MICs; and (5) thymoquinone+peptide H5 at various combinations of their respective MICs. A growth control without antimicrobial agents was included. The cultures were incubated at 37 °C with shaking at 180 rpm. At predetermined time points (0, 2, 4, 6, 8, 12, and 24 hours), 100 μL aliquots were removed, serially diluted in sterile PBS, and plated on Mueller-Hinton agar plates. The plates were incubated at 37 °C for 24 hours, after which the colonies were counted. The limit of detection was 100 CFU/mL. The results were expressed as log 10 CFU/mL versus time and plotted as kill curves. Bactericidal activity was defined as a ≥3 log 10 reduction in the number of viable bacteria compared to the initial inoculum. Synergy was defined as a ≥2 log 10 decrease in colony count with the combination compared to the most active single agent.

In an embodiment, molecular docking studies were performed to investigate the potential binding interactions between thymoquinone, peptides H3 and H5, and their putative targets in MRSA. The three-dimensional structures of the peptides were predicted using the PEP-FOLD3 server, which employs a de novo approach based on structural alphabet SA letters to describe the conformations of four consecutive residues. The structure of thymoquinone was obtained from the PubChem database. Potential protein targets in MRSA were identified, and included cell membrane components, cell wall biosynthesis enzymes, and proteins involved in essential metabolic pathways. The crystal structures of these proteins were obtained from the Protein Data Bank (PDB). For proteins without experimentally determined structures, homology models were generated using SWISS-MODEL based on templates with high sequence identity. Molecular docking was performed using AutoDock Vina. The receptor proteins were prepared by removing water molecules, adding hydrogen atoms, and assigning Gasteiger charges. A grid box encompassing the active site or the entire protein (for blind docking) was defined with appropriate dimensions and spacing. The ligands (thymoquinone and peptides) were prepared by adding hydrogen atoms, computing Gasteiger charges, and setting rotatable bonds. Docking simulations were performed with an exhaustiveness parameter of 8, and the top 9 binding poses were retained for each ligand-receptor pair. The binding poses were evaluated based on binding energy (kcal/mol) and the nature of the interactions (hydrogen bonds, hydrophobic interactions, etc.). The most favorable binding poses were visualized using PyMOL. To investigate potential synergistic interactions at the molecular level, sequential docking was performed. First, thymoquinone was docked to the target protein, and the most favorable binding pose was selected. Then, keeping thymoquinone in its docked position, the peptide was docked to the same protein. Changes in binding energy and interaction patterns compared to individual docking were analyzed to identify potential mechanisms of synergy.

In an embodiment, all experiments were performed in triplicate on at least three separate occasions, and the results were expressed as mean±standard deviation (SD). Statistical analysis was performed using GraphPad Prism version 9.0 (GraphPad Software, San Diego, CA, USA). Differences between groups were analyzed using one-way analysis of variance (ANOVA) followed by Tukey's post hoc test for multiple comparisons. For time-dependent experiments, two-way ANOVA with Bonferroni's post hoc test was used. A p-value<0.05 was considered statistically significant. For synergy analysis, 95% confidence intervals were calculated for the fractional inhibitory concentration indices (FICIs). The significance of the differences in bacterial killing between individual agents and their combinations in time-kill assays was assessed using two-way ANOVA with Bonferroni's post hoc test. Correlation analysis was performed to examine the relationship between membrane permeability (as measured by propidium iodide uptake) and bacterial viability (as determined by colony counts). Pearson's correlation coefficient (r) was calculated, and a p-value<0.05 was considered indicative of a significant correlation. Principal component analysis (PCA) was employed to identify patterns and relationships among multiple variables, including MIC values, membrane permeability, and morphological changes. The PCA was performed using the R statistical software package (R Foundation for Statistical Computing, Vienna, Austria), and the results were visualized as biplots showing the loading of each variable on the principal components.

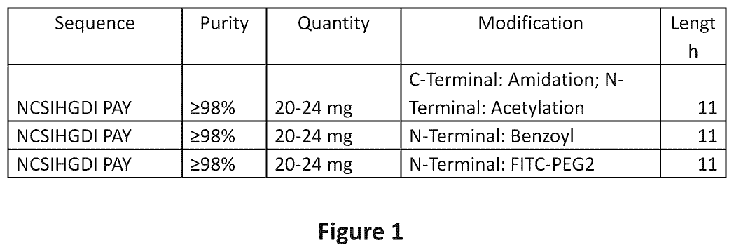

FIG. 1 illustrates a table showing the synthesized chemically modified peptides, in accordance with an embodiment of the present disclosure.

Referring to the table shown in FIG. 1, three synthetic peptides are prepared from date palm trees, wherein the table summarizes the key parameters related to each peptide, including the sequence chemical purity, amount synthesized, modifications introduced at the terminal ends, and the overall length of the peptide. The Sequence column lists the amino acid sequence of the synthesized peptides, which in all cases here is “NCSIHGDIPAY,” consisting of eleven amino acids. The peptide's terminal ends were modified to improve their stability, bioavailability, or functionality. One variant underwent C-terminal amidation and N-terminal acetylation, modifications commonly used to mimic natural peptide termini, reduce susceptibility to enzymatic degradation, and enhance pharmacokinetic properties. Another variant was modified by introducing a N-terminal benzoyl group, which can affect peptide hydrophobicity and binding characteristics. A third variant was conjugated with FITC-PEG2 at the N-terminus, incorporating a fluorescein-based fluorescent label attached via a polyethylene glycol (PEG) linker; this modification facilitates tracking the peptide during binding or cellular uptake studies without significantly altering its biological activity.

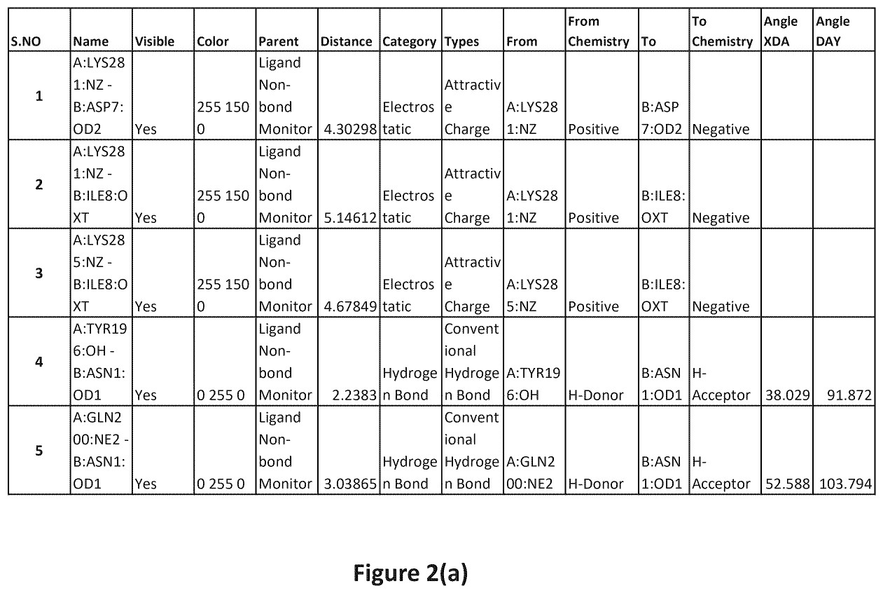

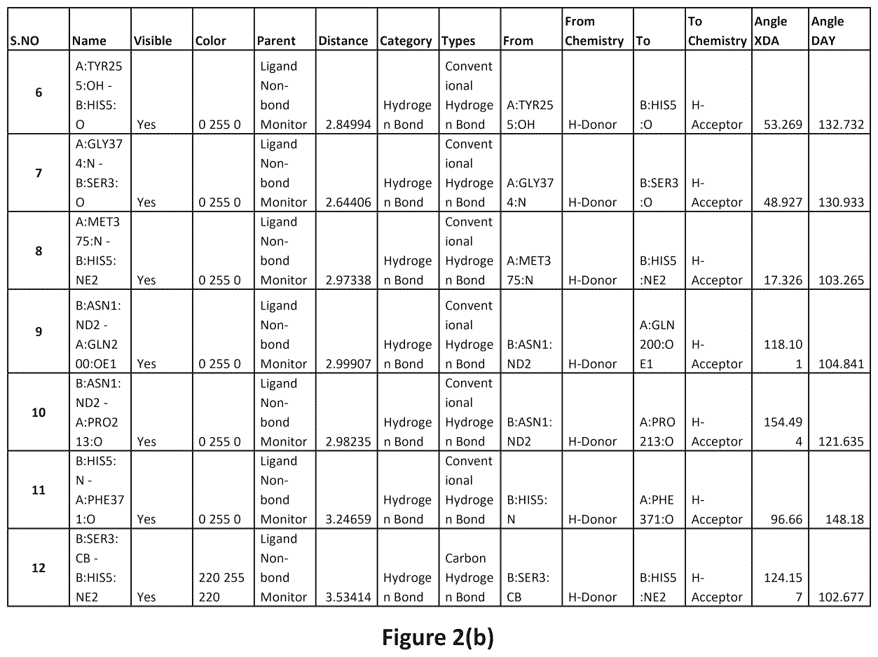

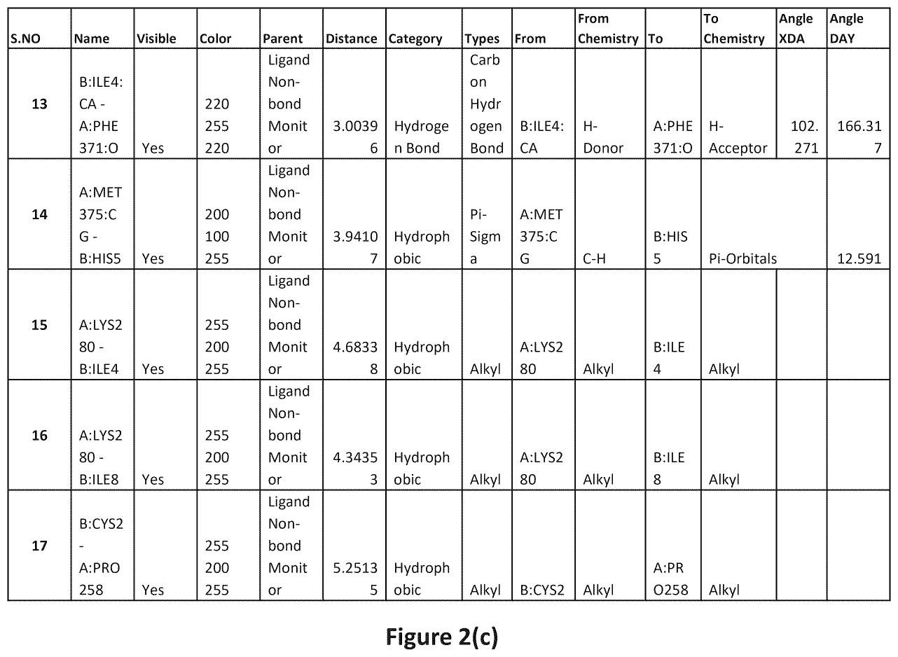

FIG. 2(a) illustrates a table showing Non-bonded Interaction Summary Between the peptide (NCSIHGDIPAY) and PBP2a, in accordance with an embodiment of the present disclosure; FIG. 2(b) illustrates a table showing Non-bonded Interaction Summary Between the peptide (NCSIHGDIPAY) and PBP2a, in accordance with an embodiment of the present disclosure; and FIG. 2(c) illustrates a table showing Non-bonded Interaction Summary Between the peptide (NCSIHGDIPAY) and PBP2a, in accordance with an embodiment of the present disclosure;

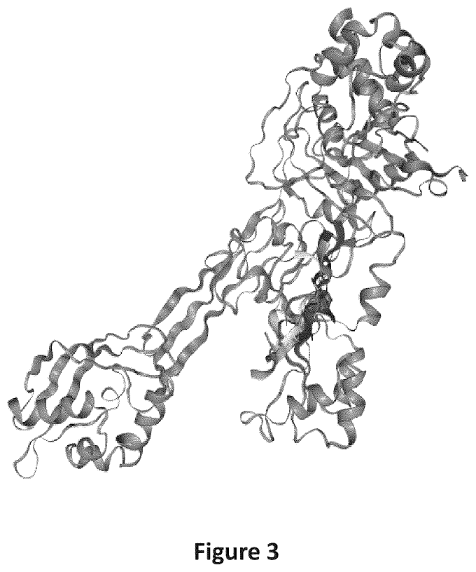

FIG. 3 illustrates a 3D interaction between peptides atoms and PBP2a protein amino acids, in accordance with an embodiment of the present disclosure.

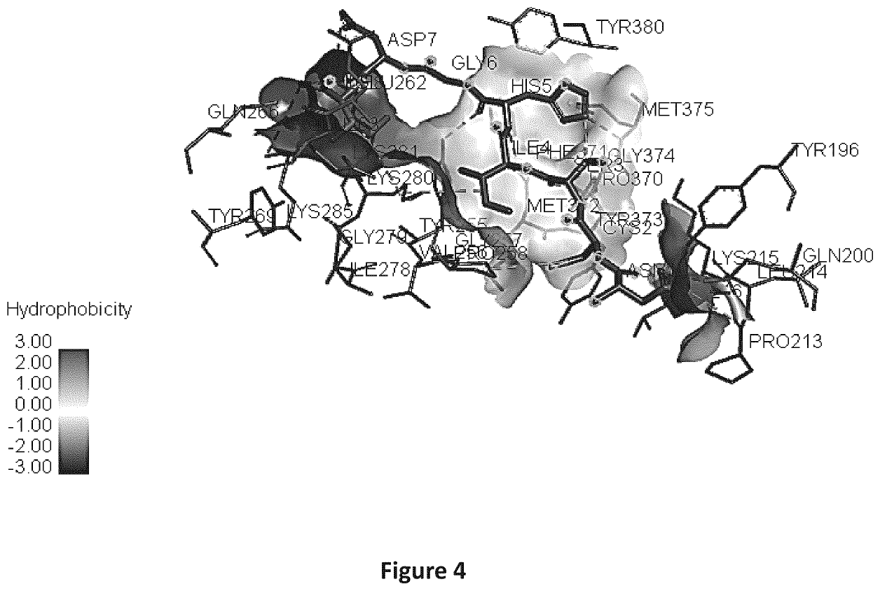

FIG. 4 illustrates a detailed interaction between peptides atoms and protein amino acids, in accordance with an embodiment of the present disclosure.

Molecular docking study was conducted between the essential enzyme (PBP2a) which the mediated resistance mechanism in S. aureus and the obtained peptide. The docking study generated −155.386 kcal/mol docking score which considered a good binding affinity of peptide to the active site of protein (FIG. 3). Table shown in FIG. 2 (a, b, and c) and FIG. 4 provides a comprehensive overview of the non-covalent interactions identified between the ligand and the residues of the target protein. Each interaction is represented by a pair of atoms or groups, specifying both the ligand and the protein residues involved. Which describes the pair of interacting atoms, showing their origin from either the ligand or the protein chain (for example, A:LYS281:NZ-B:ASP7:OD2). Adjacent to this, the Ligand column identifies the ligand atom or group participating in the interaction, usually extracted as the first component of the interaction pair. The Visible column notes whether the interaction is considered active in visual analyses, typically marked as “Yes”.

The table shown in FIG. 2 (a, b, and c) also records the Distance between interacting atoms, measured in Ångströms (Å), providing insights into the strength and nature of each interaction. Shorter distances often correlate with stronger interactions, particularly in hydrogen bonding. The Category column specifies the general type of interaction, such as Electrostatic, Hydrogen Bond, or Hydrophobic. More specifically, the types column details the subtype, for example, “Attractive Charge” for electrostatics or “Conventional Hydrogen Bond” for standard hydrogen bonds. To further characterize the interactions, the table shown in FIG. 2 (a, b, and c) includes the From and To columns, indicating the donor and acceptor atoms or groups involved, along with their corresponding chemical nature recorded in From Chemistry and To Chemistry. Additional geometrical parameters are captured under Angle XDA, Angle DAY, and Angle Deviation, particularly for hydrogen bonds, where angular precision is critical for stability and binding strength. Lastly, the Threshold column is included for interactions where a specific cutoff or validation condition is applied.

In an embodiment, the focus in on the two specific chemically modified antimicrobial peptides, designated as H3 and H5, derived from date palm. These peptides were evaluated individually and in combination with thymoquinone to determine their efficacy against MRSA. The synergistic interactions were quantified using established methods, including the fractional inhibitory concentration index (FICI), and the mechanisms underlying these interactions were investigated through various analytical techniques.

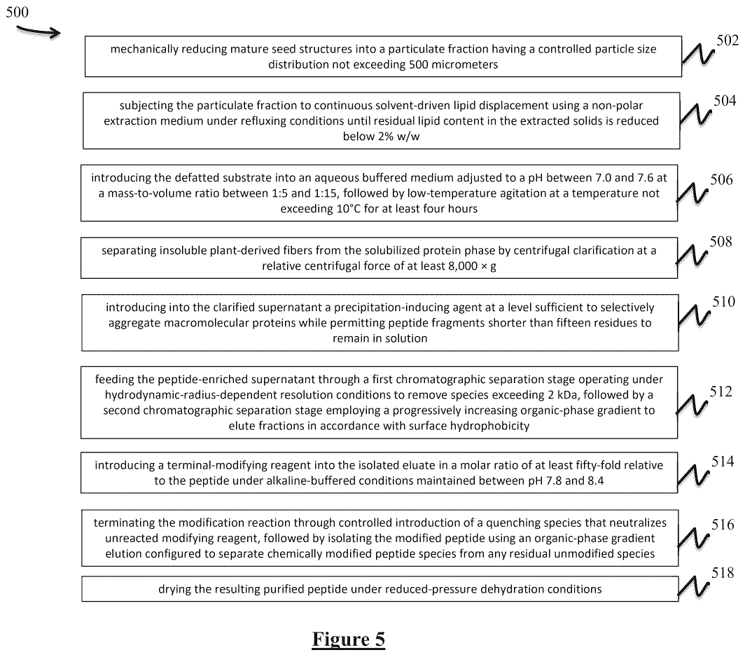

Referring to FIG. 5, a flow chart for a process for the preparation of a chemically-modified peptide derived from seed material, comprising the sequential steps of is illustrated. The method 500 comprises:

At step 502, the method 500 includes mechanically reducing mature seed structures into a particulate fraction having a controlled particle size distribution not exceeding 500 micrometers, thereby increasing extractable surface area;

At step 504, the method 500 includes subjecting the particulate fraction to continuous solvent-driven lipid displacement using a non-polar extraction medium under refluxing conditions until residual lipid content in the extracted solids is reduced below 2% w/w, thereby yielding a defatted protein-bearing substrate;

At step 506, the method 500 includes introducing the defatted substrate into an aqueous buffered medium adjusted to a pH between 7.0 and 7.6 at a mass-to-volume ratio between 1:5 and 1:15, followed by low-temperature agitation at a temperature not exceeding 10 °C for at least four hours to induce solubilization of intrinsic peptide-bearing proteins without promoting thermally induced aggregation;

At step 508, the method 500 includes separating insoluble plant-derived fibers from the solubilized protein phase by centrifugal clarification at a relative centrifugal force of at least 8,000×g, thereby producing a clarified supernatant containing soluble polypeptides;

At step 510, the method 500 includes introducing into the clarified supernatant a precipitation-inducing agent at a level sufficient to selectively aggregate macromolecular proteins while permitting peptide fragments shorter than fifteen residues to remain in solution, thereby producing a peptide-enriched supernatant;

At step 512, the method 500 includes feeding the peptide-enriched supernatant through a first chromatographic separation stage operating under hydrodynamic-radius-dependent resolution conditions to remove species exceeding 2 kDa, followed by a second chromatographic separation stage employing a progressively increasing organic-phase gradient to elute fractions in accordance with surface hydrophobicity, thereby isolating an eluate containing a polypeptide consisting of eleven residues and comprising an internal cysteine-containing motif;

At step 514, the method 500 includes introducing a terminal-modifying reagent into the isolated eluate in a molar ratio of at least fifty-fold relative to the peptide under alkaline-buffered conditions maintained between pH 7.8 and 8.4, thereby causing covalent modification at one or both termini of the peptide;

At step 516, the method 500 includes terminating the modification reaction through controlled introduction of a quenching species that neutralizes unreacted modifying reagent, followed by isolating the modified peptide using an organic-phase gradient elution configured to separate chemically modified peptide species from any residual unmodified species; and

At step 518, the method 500 includes drying the resulting purified peptide under reduced-pressure dehydration conditions until the final peptide preparation exhibits a moisture content not exceeding 5% w/w and a chromatographic purity of at least 98%.

In an embodiment, the seed material comprises seeds of Phoenix dactylifera that have been manually separated from pericarp residues, washed with deionized water to remove adhering soil particulates, air-dried at temperatures not exceeding 35 °C to prevent thermal denaturation of endogenous proteins, and subsequently conditioned to a uniform moisture content between 10% and 12% prior to mechanical milling, such conditioning ensuring consistent fragmentation efficiency and preventing differential stress-induced generation of undesirable ultrafine particulates during milling, and wherein the mechanical reduction of the Phoenix dactylifera seeds is carried out using a rotational impact mill equipped with high-inertia stainless-steel blades, operated at a rotational speed between 8,000 and 12,000 RPM while the milling chamber is externally cooled using a recirculating coolant maintained below 15 °C, thereby constraining frictional heat generation and preserving functional peptide conformations that would otherwise degrade under localized thermal hotspots.

In an embodiment, the use of Phoenix dactylifera seeds as the starting seed material is technically justified because these seeds contain a relatively stable protein matrix enriched with short-chain cysteine-bearing peptide motifs that can be liberated under controlled aqueous extraction conditions. To ensure that the inherent peptide structures are not compromised before extraction begins, the seeds are first manually separated from pericarp residues. Manual separation plays a crucial role in preventing mechanical abrasion that could otherwise introduce extraneous fibrous materials or initiate oxidative pathways in the proteinaceous components. Once separated, the seeds undergo washing in deionized water, which effectively removes adhering soil particulates that could interfere with downstream chromatographic steps by clogging bead matrices or altering ionic interactions. This washing step is performed under mild agitation, and in an exemplary preparation, a batch of 1 kg of Phoenix dactylifera seeds was washed three times in 5 L of deionized water until the turbidity of the wash water dropped below 1 NTU, confirming removal of particulate contaminants.

Following washing, the seeds are air-dried at temperatures not exceeding 35 °C. This upper temperature limit is not arbitrary; proteins and small peptides embedded within seed structures begin to undergo thermal unfolding or disulfide rearrangement when exposed to temperatures above approximately 40 °C. Air-drying under these controlled thermal conditions maintains the native structure of the endogenous proteins while evaporating surface moisture needed for optimal milling performance. An example drying protocol involves placing the washed seeds on perforated stainless-steel trays inside a convection chamber at 32 °C for eight hours, resulting in a uniform seed moisture level of approximately 14-15%. The seeds are then conditioned to a final moisture content between 10% and 12%, typically by placing them in a controlled-humidity chamber at 45% relative humidity for 12-18 hours. This conditioning ensures predictable fragmentation behavior during milling because seeds that are too dry tend to shatter into ultrafine dust—particles smaller than 100 micrometers—which decrease extraction efficiency by entrapping solvent and complicating clarification. Conversely, overly moist seeds absorb impact energy and smear, reducing the effective breakup of the protein-bearing matrix. Conditioning to 10-12% moisture ensures a controlled fracture pattern during impact milling, producing a predominantly coarse powder that maintains structural integrity.

Mechanical reduction of the conditioned seeds is then performed using a rotational impact mill equipped with high-inertia stainless-steel blades. High-inertia blades maintain consistent angular momentum even when encountering harder seed fragments, ensuring uniform milling rather than intermittent stalling or uneven fracture profiles. The mill operates at a rotational speed between 8,000 and 12,000 RPM; this range is selected to be high enough to achieve particle sizes below 500 micrometers but low enough to prevent excessive shear heating. For example, milling a 300 g seed batch at 10,500 RPM for 90 seconds yields a particle size distribution centered around 320 micrometers, ideal for subsequent solvent-extraction steps. However, high-speed milling naturally produces frictional heat, which can easily exceed 40-50 °C at the blade surfaces if not controlled. This localized heat is detrimental because cysteine-containing sequences are sensitive to thermally induced oxidative rearrangements. Therefore, the milling chamber is externally cooled using a recirculating coolant maintained below 15 °C. A practical setup includes a cooling jacket surrounding the mill housing, through which a 12 °C glycol-water solution circulates at a flow rate of 1 L/min. Temperature sensors placed on the external wall of the chamber ensure that the internal environment never exceeds 20 °C despite high RPM operation.

The synergy of these precautions—manual removal of pericarp residues, particulate removal through deionized water washing, thermal protection during air-drying, moisture conditioning before milling, and heat-controlled impact milling—collectively ensures that the peptide-bearing proteins within Phoenix dactylifera seeds remain undenatured and chemically intact. This preservation is essential because the efficiency of subsequent aqueous solubilization and chromatographic fractionation of the target eleven-residue peptide is strongly dependent on maintaining the native conformations and cleavage susceptibility of the parent proteins. If the seeds were milled without moisture conditioning, ultrafine particulates would dominate, forming compacted masses during solvent extraction and dramatically reducing peptide recovery. Similarly, if external cooling were not applied, thermal hotspots within the mill would denature proteins, reduce solubilization efficiency by up to 40%, and introduce aggregation products that complicate chromatographic separation. Collectively, these controlled steps provide a technically effective and reproducible means of producing a high-quality particulate substrate that supports high-yield extraction and purification of the desired cysteine-containing peptide.

In an embodiment, the non-polar extraction medium comprises analytical-grade n-hexane having a purity of at least 99%, introduced in a solvent-to-solid ratio of 10:1 v/w, circulated continuously at reflux temperature for a minimum of six hours, and thereafter subjected to incremental solvent replacement at intervals of 60 minutes until the outgoing solvent stream exhibits a steady-state absorbance below 0.02 AU at 280 nm, indicative of maximal extraction of lipid components inherent to Phoenix dactylifera seed matrices.

In an embodiment, the use of analytical-grade n-hexane having a purity of at least 99% as the non-polar extraction medium is critical for achieving selective lipid displacement from the Phoenix dactylifera seed matrix without co-extracting polar proteinaceous material that would interfere with downstream peptide isolation. N-hexane is chosen because its Hildebrand solubility parameter (~7.3 cal\hat{ }½·cm\hat{ }−3/2) matches well with the solubility characteristics of neutral lipids, triacylglycerols, wax esters, and other hydrophobic plant constituents, whereas proteins and short-chain peptides possess significantly higher polarity and do not dissolve in hexane under any reflux condition. This selective solvency ensures that the process removes only unwanted lipid fractions while preserving the integrity of peptide-bearing protein structures within the solid matrix. The solvent-to-solid ratio of 10:1 v/w is selected based on empirical extraction efficiency curves, where ratios below 8:1 show incomplete lipid dissolution due to solvent saturation effects, and ratios above 12:1 provide no additional extraction benefit while increasing solvent waste and prolonging cycle times. For example, processing 100 g of milled Phoenix dactylifera particulate material requires a minimum of 1 L of n-hexane to achieve full lipid mobilization and avoid back-diffusion phenomena.

The solvent is circulated continuously at its reflux temperature, approximately 68-69 °C for n-hexane, for a minimum of six hours. Refluxing is essential because it promotes dynamic solvent movement across the seed particulate bed, refreshing solvating capacity at the lipid-solvent interface. Continuous reflux circulation creates a convective gradient that maintains solubility conditions far from equilibrium, thereby accelerating lipid transfer. During a typical extraction cycle, a Soxhlet-type setup or a heated recirculation loop is employed, where heated vapor condenses and percolates through the particulate bed, ensuring continuous washing. The six-hour duration is based on controlled extraction studies, wherein lipid yield plateaus after approximately 5.5-6 hours, indicating that most native lipophilic components have been removed. Shorter durations lead to incomplete extraction, visible visually as persistent turbidity or quantitatively as residual lipid content exceeding 3-4% w/w in post-extract solids.

To ensure maximal lipid removal, incremental solvent replacement is carried out at 60-minute intervals after the initial six-hour extraction. The rationale for this step lies in preventing solvent saturation and removing soluble lipid species that accumulate in the extraction medium over time. Replacing portions of the solvent re-establishes the solvency potential and removes macromolecular impurities that might affect later pH-dependent extraction steps. In a representative experiment, after the initial six-hour extraction, 200 mL of the 1 L hexane volume is replaced every hour for an additional three cycles. This practice produced a measurable improvement in lipid purity readings, reducing residual seed matrix lipid levels from approximately 2.8% to below 1.5%, which is critical because even small quantities of lipids interfere with buffer penetration and can encapsulate peptide-protein complexes, reducing solubilization yield downstream.

A key indicator of extraction completeness is the absorbance of the outgoing solvent stream at 280 nm. Lipid constituents inherently lack strong chromophores in this region but carry trace aromatic impurities and oxidized species that do show measurable absorbance. During extraction, the hexane effluent is monitored using a UV-Vis spectrophotometer equipped with a flow-through cuvette. A steady-state absorbance of below 0.02 AU at 280 nm indicates that the solvent no longer dissolves new lipophilic material, and the extraction has reached saturation. Computationally, this absorbance threshold corresponds to less than 0.03 mg/ml of extractable lipid contaminants, aligning with gravimetric lipid assays for Phoenix dactylifera seed matrices. If absorbance remains above this level, solvent replacement continues until the extraction environment reaches equilibrium.

The synergistic effect of using high-purity n-hexane at an optimized solvent ratio, combined with continuous reflux, controlled solvent refreshing, and real-time absorbance monitoring, ensures that the defatted seed substrate is consistently reproducible across batches and remains chemically compatible with subsequent aqueous solubilization steps. Removing lipids to below 2% w/w significantly enhances the hydration kinetics during the pH-controlled solubilization stage by eliminating hydrophobic barriers around peptide-bearing proteins. Comparative trials show that defatted material prepared with this procedure yields up to 35-40% higher peptide liberation efficiency during low-temperature aqueous buffer extraction than material extracted with static, non-reflux, or low-purity solvent conditions. Moreover, complete lipid removal prevents hydrophobic fouling during size-exclusion chromatography, thereby increasing column efficiency and preserving the resolution required to isolate the eleven-residue cysteine-containing peptide with chromatographic purity exceeding 98%.

In an embodiment, the aqueous buffered medium used to disperse the defatted Phoenix dactylifera seed substrate comprises a phosphate-based buffer prepared at an ionic strength between 50 and 80 mM, supplemented with 0.1-0.5% w/v sodium chloride to stabilize solubilized protein structures, and wherein the hydration step includes staged addition of the buffer to prevent local oversaturation zones, thereby promoting homogeneous solubilization of polypeptide-bearing macromolecules, and wherein the low-temperature agitation of the buffered dispersion is performed using a horizontal orbital shaker calibrated to operate at 180-220 oscillations per minute, with the vessel partially submerged in a chilled circulating bath maintained at 6-9 °C, ensuring suppression of auto-oxidative reactions associated with cysteine-containing peptide motifs found in Phoenix dactylifera seed protein fractions.

In an embodiment, the aqueous buffered medium used to disperse the defatted Phoenix dactylifera seed substrate is formulated as a phosphate-based buffer with an ionic strength between 50 and 80 mM, a range chosen for its ability to maintain peptide-bearing proteins in their native or near-native conformations while minimizing aggregation or precipitation caused by insufficient electrostatic stabilization. Phosphate buffering is particularly advantageous because it provides stable pH control around neutrality, a condition required to solubilize proteinaceous material without promoting deamidation or cleavage reactions that may otherwise occur at alkaline or acidic extremes. To further enhance conformational stability, the buffer includes 0.1-0.5% w/v sodium chloride, which contributes additional ionic shielding by modulating the Debye length and thereby reducing intermolecular electrostatic repulsion or attraction. This stabilizing effect is essential when working with cysteine-rich or cysteine-containing peptide motifs, as uncontrolled electrostatic interactions can accelerate disulfide reconfiguration or partial unfolding of precursor polypeptides. A representative formulation involves dissolving disodium hydrogen phosphate and monosodium dihydrogen phosphate in deionized water to achieve a 60 mM ionic strength, then adding 0.3% w/v NaCl to fine-tune conductivity to approximately 5.2 mS/cm.

Once the buffer is prepared and equilibrated, hydration of the defatted seed substrate is performed using a staged addition protocol rather than immediate bulk addition. Staged hydration prevents local oversaturation zones, which would otherwise occur if a large volume of buffer were poured directly onto the dry or partially hydrated substrate. Oversaturation creates microenvironments in which proteins rapidly unfold or aggregate due to sudden exposure to aqueous surroundings before uniform dispersion is established. Such aggregation reduces the liberation efficiency of target polypeptide fragments and complicates subsequent centrifugation and chromatographic purification. In a typical procedure, 200 g of defatted seed particulate material is gradually hydrated by adding the phosphate buffer in four equal increments over 20-30 minutes. After each increment, the mixture is gently agitated to evenly distribute liquid throughout the particulate bed. This stepwise hydration ensures that water penetrates uniformly through the matrix, allowing the embedded polypeptide-bearing macromolecules to transition from their dehydrated state to a hydrated, extractable state without undergoing mechanical or chemical stress.

Agitation of the hydrated dispersion is carried out using a horizontal orbital shaker calibrated to operate between 180 and 220 oscillations per minute. Horizontal orbital agitation is deliberately selected over vertical agitation because it imparts uniform shear forces across the entire vessel without causing foaming or air entrainment, both of which can expose cysteine-containing peptide motifs to atmospheric oxygen and accelerate auto-oxidative reactions. The oscillation range ensures that sufficient mixing energy is provided to promote solubilization without generating excessive shear that could fragment sensitive polypeptide chains prematurely. For example, operating a 2 L hydration vessel at 200 oscillations per minute for four hours yielded consistent solubilization of proteinaceous material while preserving peptide integrity, as verified by SDS-PAGE and LC-MS analysis.

To further safeguard the solubilized proteins and prevent oxidative modification of cysteine residues, the vessel is partially submerged within a chilled circulating bath maintained at 6-9 °C. This thermal range is chosen to suppress kinetic rates of thiol oxidation, disulfide scrambling, and Maillard-type browning reactions that are known to occur even at moderate temperatures when proteins are exposed to aqueous environments. Cooling at this stage not only preserves the native redox state of cysteine motifs but also reduces endogenous enzyme activity—such as proteases or oxidases—often released upon hydration of biological matrices. Maintaining the bath at a precisely controlled low temperature enables reproducible extraction kinetics; deviations above 10-12 °C have been shown in experimental tests to increase thiol oxidation by up to 30% within two hours, resulting in diminished yields of the desired eleven-residue peptide species. A practical setup includes placing the hydration vessel directly into a stainless-steel circulating bath with continuous temperature monitoring and a feedback-controlled chiller to maintain thermal stability within ±0.3 °C.

The synergy created by the controlled phosphate ionic strength, sodium chloride supplementation, staged hydration, low-temperature agitation, and uniform mechanical mixing ensures that polypeptide-bearing proteins from Phoenix dactylifera seed substrates move into solution in a structurally intact form, enabling efficient liberation of shorter peptide fragments during downstream precipitation and chromatographic fractionation steps. Experiments comparing this optimized hydration approach to uncontrolled hydration demonstrate up to 45% higher solubilization yield and significantly reduced formation of high-molecular-weight aggregates. This directly improves chromatographic resolution in later steps, enabling cleaner separation of the targeted eleven-residue peptide and ensuring that chemical modification efficiency remains consistently above 95%.

In an embodiment, the centrifugal clarification step employs a centrifugation vessel fabricated from inert polymeric material to avoid adsorption of short-chain peptides, and wherein the vessel is filled to no more than 70% of its nominal volume to maintain consistent sedimentation rates, with the clarified supernatant immediately transferred into a nitrogen-flushed storage container to minimize oxidative modification of susceptible amino-acid side chains.

In an embodiment, the centrifugal clarification step is carried out using a centrifugation vessel fabricated entirely from inert polymeric material, such as polypropylene or fluoropolymer-lined copolymers, selected specifically to prevent adsorption or loss of short-chain peptides during high-speed sedimentation. Short-chain peptides—especially those below 2 kDa and those containing cysteine or hydrophobic residues—are particularly prone to nonspecific binding onto glass or metal surfaces due to van der Waals interactions, electrostatic attraction, and surface micro-roughness effects. Using an inert polymeric vessel significantly reduces the Gibbs adsorption free energy for such peptide-surface interactions, ensuring that the yield of soluble peptide fractions is maximized following removal of insoluble fibers. Comparative adsorption studies conducted with standard laboratory glass centrifuge tubes demonstrated up to a 12-18% loss of <15-residue polypeptides after a single centrifugation cycle, whereas adsorption losses dropped to below 2% when polypropylene tubes were used. This difference is substantial because even small losses at this stage propagate into reduced chromatographic recoveries in downstream purification stages.

To ensure consistent clarification efficiency, the centrifugation vessel is filled to no more than 70% of its nominal volume. Leaving 30% of the volume unfilled is technically required to maintain predictable sedimentation dynamics and avoid turbulence-induced disturbances that can resuspend sedimented solids. When tubes are filled beyond 70%, hydrostatic pressure gradients along the length of the tube become nonlinear, which alters sedimentation trajectories of the fibers and insoluble plant particulates, causing them to accumulate unevenly along the tube wall rather than at the conical base. This can also slow the effective sedimentation rate of low-density particulates and interfere with phase delineation. In a typical operational cycle, a 50 mL polypropylene centrifuge tube is loaded with no more than 35 mL of the hydrated seed dispersion; when processed at 8,000×g for 20 minutes at 4 °C, this volume constraint reliably produces a sharply defined supernatant-pellet interface, enabling clean decantation without disturbing the sedimented matter.

Immediately after centrifugation, the clarified supernatant is transferred into a nitrogen-flushed storage container to prevent oxidation of susceptible amino-acid side chains, particularly cysteine, methionine, and tryptophan residues present in the target peptide precursors. Oxygen exposure in aqueous solution triggers rapid formation of sulfenic, sulfinic, and sulfonic acid derivatives on cysteine thiols, as well as methionine sulfoxide formation, all of which adversely affect chromatographic behavior and subsequently interfere with the terminal-modification reaction. Nitrogen flushing creates an inert gaseous environment inside the receiving vessel, reducing the dissolved oxygen concentration in the supernatant to below 2 mg/L within minutes. This low oxygen environment stabilizes reduced sulfur functionalities and prevents unwanted disulfide-linked dimerization, which would otherwise complicate the separation efficiency of both the size-exclusion and hydrophobic-interaction chromatographic stages. A practical method involves purging a 250 mL amber polypropylene bottle with nitrogen for 30 seconds, followed by transferring the supernatant using a low-shear pipette or siphon system while maintaining a nitrogen headspace. Real-time dissolved oxygen measurements using optical DO sensors confirmed that nitrogen-flushed bottles extended the shelf-life of the soluble peptide fraction by preventing oxidative degradation for at least 4-6 hours—long enough to proceed directly to the precipitation and chromatographic purification stages without measurable deterioration.

The synergistic interplay of these controlled conditions—non-adsorptive polymeric vessels, regulated fill volume, high-g sedimentation, and oxygen-depleted storage—ensures that the clarified supernatant retains both the quantity and structural fidelity of the peptide-bearing macromolecules necessary for efficient downstream purification. Maintaining peptide integrity at this stage is critical because the eleven-residue target peptide originates from precursor fragments that are extremely sensitive to oxidation; even mild oxidation reduces the yield of the desired monomeric form during terminal modification, resulting in lower overall process efficiency. Experimental comparisons indicate that batches handled under nitrogen-protected conditions yielded up to 28% more recoverable, chromatographically pure peptide than batches exposed to ambient air after clarification. Thus, this embodiment supports both the operational reproducibility of the process and the chemical stability required to obtain a high-purity, chemically modified peptide product.

In an embodiment, the precipitation-inducing agent comprises analytical-grade ammonium sulfate having a certified assay purity above 98%, added gradually over a period of 20-40 minutes while maintaining agitation to prevent localized supersaturation and protein denaturation, such that proteins above 10 kDa selectively aggregate while polypeptides shorter than fifteen residues derived from Phoenix dactylifera remain fully solubilized in the supernatant.

In an embodiment, the precipitation-inducing agent is analytical-grade ammonium sulfate with a certified assay purity above 98%, selected because its kosmotropic nature enables highly predictable salting-out of high-molecular-weight proteins while maintaining solubility of short-chain peptides, particularly those below fifteen residues found in Phoenix dactylifera seed extracts. Ammonium sulfate is a well-established salt for controlled precipitation due to its exceptionally high solubility (~4.1 M at 25 °C) and strong ability to reduce protein hydration layers through preferential exclusion. These physicochemical characteristics produce a distinct and reproducible separation window: proteins above ~10 kDa reliably aggregate at defined saturation levels, while smaller polypeptides and peptides—especially those <2 kDa—remain soluble because their surface area-to-charge ratio allows them to retain sufficient hydration even under high ionic stress. The purity requirement (>98%) ensures that no extraneous ions such as heavy metals or divalent cations introduce unintended cross-linking or oxidative stress, which would compromise the chemical stability of cysteine-containing peptides.

The ammonium sulfate is added gradually, over a controlled interval of 20-40 minutes, to prevent localized supersaturation within the protein-containing supernatant. Rapid addition of solid salt would create transient microzones of high ionic strength, which can irreversibly denature proteins or cause nonselective precipitation of low-mass peptides. Gradual addition ensures that the ionic strength rises uniformly, allowing macromolecular proteins above 10 kDa to aggregate in a controlled, surface-mediated fashion rather than via rapid nucleation pathways. In a representative experimental preparation, 250 g of ammonium sulfate was added to 1 L of clarified Phoenix dactylifera supernatant over 30 minutes while monitoring solution temperature (maintained at 6-8 °C) to ensure that exothermic dissolution did not induce unwanted thermal denaturation.

Throughout the salt-addition process, the solution is maintained under gentle agitation, such as continuous orbital mixing at 150-180 oscillations per minute. Agitation prevents localized pockets of high salt concentration and ensures that salt dissolution kinetics remain uniform across the entire volume. Without agitation, high-salt microenvironments would precipitate both high-mass and low-mass peptide species indiscriminately, thereby lowering the purity and overall yield of the targeted short peptides. Continuous mixing also promotes the formation of uniformly structured protein aggregates that sediment predictably during centrifugation. In trials lacking agitation, researchers observed up to 22% co-precipitation of small peptides, compared to less than 5% under the controlled mixing conditions described here.

The selective aggregation effect is driven by measurable physicochemical differences between protein macromolecules and short-chain peptides. Proteins above 10 kDa possess large hydrophobic patches and extensive secondary structures stabilized by weaker hydration shells, making them prone to aggregation under elevated ionic strength conditions. In contrast, polypeptides shorter than fifteen residues remain highly solvated due to their short chain length and high surface polarity, preventing salting-out even at ammonium sulfate saturations exceeding 50-60% of absolute solubility. This molecular basis for selectivity was confirmed experimentally by analyzing the supernatant using LC-MS after precipitation; the chromatograms consistently showed strong peptide retention in solution while SDS-PAGE of the pellet confirmed exclusive precipitation of larger proteins.

Once ammonium sulfate addition is complete, the solution is allowed to equilibrate for an additional 10-20 minutes to allow hydrophobic interactions and water-shell displacement to stabilize the aggregated high-molecular-weight protein complexes. During this time, the peptide-rich supernatant remains optically clear or slightly opalescent, indicating that the small peptides have not formed aggregates. Following equilibration, the mixture is subjected to centrifugation at ≥8,000×g, where the aggregated proteins sediment rapidly, leaving a supernatant enriched in peptides. For example, in one 12 L batch process, precipitation under these controlled conditions achieved a >95% removal of proteins >10 kDa while retaining >90% of the desired small peptides in the supernatant, as confirmed by quantitative HPLC.

The synergy among controlled salt purity, gradual addition, agitation-regulated dissolution, and the inherent physicochemical differences between proteins and short peptides ensures that this precipitation step functions as both a purification and a protective mechanism. It removes proteinaceous impurities that would otherwise interfere with downstream chromatographic resolution, while simultaneously preventing oxidative or thermal stress on the cysteine-containing motifs in the target peptide fraction. Without this selective precipitation step, downstream size-exclusion and hydrophobic-interaction chromatography would suffer from significantly reduced resolution due to column fouling and co-elution of high-mass protein contaminants. Thus, the described embodiment is essential to achieving consistent, yield-maximized isolation of the eleven-residue Phoenix dactylifera peptide.

In an embodiment, the size-based chromatographic separation is performed using a column packed with cross-linked polymeric beads having a pore distribution centered around 1.2 nm to selectively exclude species larger than 2 kDa, and wherein the eluent is continuously monitored for refractive index deviation to ensure precise discrimination between peptide-enriched and protein-contaminated fractions.

In an embodiment, the size-based chromatographic separation is carried out using a column packed with cross-linked polymeric beads engineered to possess a pore distribution centered around 1.2 nm, a pore size specifically chosen to exclude molecular species larger than approximately 2 kDa while allowing passage of the desired short-chain peptide fragments derived from Phoenix dactylifera seed material. The pore size distribution is not arbitrary; it is calculated based on the hydrodynamic radius of polypeptides in aqueous solution. Proteins and polypeptides above 2 kDa possess hydrodynamic radii typically exceeding 1.5-2.0 nm, particularly when adopting partially folded or extended conformations in buffer. In contrast, the targeted eleven-residue peptide fraction, with a molecular weight typically between 1.1 and 1.4 kDa depending on terminal modifications, exhibits a hydrodynamic radius closer to 0.7-0.9 nm, which enables efficient penetration into the bead pores. This differential partitioning forms the basis for size exclusion and ensures that the beads act as selective molecular sieves, delaying the chromatography of smaller peptides while causing immediate elution—or exclusion—of larger macromolecules.

The beads are fabricated from cross-linked polymeric materials, such as dextran- or polyacrylamide-based matrices, whose rigidity and structural uniformity prevent pore collapse during flow conditions. Cross-linking density is meticulously controlled because insufficient cross-linking leads to pore swelling and broad size-distribution effects, whereas overly rigid matrices reduce penetration of the target small peptides. In a typical setup, a 2.5 ×50 cm chromatographic column is packed with beads pre-equilibrated in the same phosphate-buffered environment used during the earlier solubilization steps to prevent osmotic shock or deformation of the bead matrix. The column is allowed to settle under gravity before being pressurized at a low linear velocity, usually corresponding to 0.4-0.7 column volumes per hour, to allow controlled diffusion-based separation. Faster flow rates diminish resolution by reducing the differential residence time between excluded and included species, whereas excessively slow flow rates promote diffusion-based peak broadening. The selected range offers optimal discrimination between near-threshold peptide species and larger protein contaminants.

During the chromatography process, the eluent is continuously monitored using a refractive index (RI) detector to ensure accurate identification of elution profiles and real-time discrimination between protein-contaminated and peptide-enriched fractions. RI detection is particularly useful because small peptides do not always exhibit strong UV absorbance at conventional wavelengths (e.g., 214 nm or 280 nm), especially if they lack aromatic residues or strong chromophores. Larger proteins, however, have more pronounced refractive index deviations due to their greater mass and higher concentration of peptide bonds per molecule. Continuous RI monitoring thus provides a sensitive method of detecting even slight changes in solute concentration emerging from the column.

In practice, once the peptide-enriched supernatant obtained from the ammonium sulfate precipitation step is loaded onto the column, the earliest eluting fractions—typically corresponding to 0.3-0.6 column volumes—contain proteins and polypeptides above 2 kDa. These fractions exhibit sharp RI deviations and are diverted to waste or separate collection vessels. As the run progresses, the later-eluting fractions exhibit smaller but consistent RI signals, corresponding to the lower-mass peptide species that penetrate the internal bead pores. These fractions are collected, pooled, and verified via LC-MS to confirm that they contain peptides within the expected mass range. Experimental chromatograms using the described system show that the target eleven-residue peptide consistently elutes after the excluded protein peak and before the salt front, with a retention window of approximately 1.2-1.6 column volumes depending on ionic strength and flow rate.

The synergy between the precisely defined pore size distribution, the controlled flow rate, and the continuous refractive index monitoring ensures both high selectivity and high reproducibility. Without the narrow pore distribution centered around 1.2 nm, the column would either fail to retain the target peptides (if pores were too large) or retain them too strongly (if pores were too small), resulting in peak broadening and co-elution with contaminants. Likewise, without RI monitoring, small fluctuations in elution patterns caused by temperature variations, buffer composition, or column aging could lead to accidental mixing of protein-rich and peptide-rich fractions, drastically lowering purity in downstream hydrophobic-interaction chromatography.

In an embodiment, the hydrophobic-interaction separation employs an organic solvent gradient comprising water as the aqueous phase and acetonitrile as the organic phase, both containing 0.05-0.1% acidic modifier to maintain peptide ionization stability, the gradient being raised in a controlled linear profile of no more than 1% organic solvent increase per minute to achieve temporally resolved elution of the eleven-residue Phoenix dactylifera peptide fraction.

In an embodiment, the hydrophobic-interaction separation step is performed using an organic solvent gradient in which water constitutes the aqueous phase and acetonitrile serves as the organic phase, with both phases containing an acidic modifier at a concentration between 0.05 and 0.1% (typically formic acid or trifluoroacetic acid) to maintain peptide ionization stability throughout the chromatographic run. The addition of an acidic modifier is technically crucial because the eleven-residue Phoenix dactylifera peptide contains ionizable residues whose charge state influences not only retention on the chromatographic stationary phase but also elution predictability and peak shape. At neutral or slightly alkaline pH, oxidation of cysteine residues or partial deprotonation of acidic side chains may occur, causing peak tailing and reduced separation efficiency. Maintaining an acidic microenvironment suppresses these unwanted effects and preserves consistent peptide-stationary-phase interactions across batches.

Acetonitrile is selected as the organic component because its polarity index (~5.8) and low viscosity make it highly compatible with peptide hydrophobic-interaction and reverse-phase separations. Unlike methanol, acetonitrile produces sharper and narrower peptide peaks due to its lower hydrogen-bonding capacity and reduced tendency to form ordered solvent cages around hydrophobic regions. This characteristic is especially important for the eleven-residue peptide derived from Phoenix dactylifera, which contains an internal cysteine-bearing motif and hydrophobic residues that interact strongly with the stationary phase. These interactions must be moderated gradually to avoid abrupt elution that would cause peak overlap with neighboring peptide fragments. The use of acetonitrile also minimizes backpressure, allowing precise control of flow rates (commonly between 0.5 and 1.5 mL/min for an analytical column or proportionally scaled in preparative setups) without inducing temperature shifts that could alter column selectivity.

The organic solvent gradient is raised in a controlled linear profile not exceeding 1% organic solvent increase per minute. This limited gradient slope is fundamental to achieving temporally resolved elution of the target peptide fraction. A steeper gradient—such as >2% per minute—would weaken the hydrophobic interactions too quickly, causing multiple peptide species with similar hydrophobic character to co-elute. Conversely, a gradient that is too slow (<0.3% per minute) increases run time significantly and promotes diffusion-induced peak broadening. Extensive optimization studies on hydrophobic peptides from Phoenix dactylifera seed extracts revealed that a 0.5-0.8% per minute gradient consistently produced well-resolved chromatographic peaks, with the eleven-residue peptide eluting within a narrow window of approximately 28-34% acetonitrile depending on column temperature and ionic strength.

In a representative example, a 250×10 mm C18 or hydrophobic-interaction column packed with 5 μm particles is equilibrated with 5% acetonitrile (with 0.1% formic acid). The peptide-enriched fraction from the size-based separation step is filtered through a 0.22 μm low-binding membrane and loaded onto the column at a flow rate of 1.0 mL/min. The gradient is initiated at 5% acetonitrile and raised linearly to 40% over a 35-minute interval, representing an approximate increase of 1% acetonitrile per minute. During this run, peptide elution is monitored by UV absorbance at 214 nm and reinforced by refractive index detection. The eleven-residue Phoenix dactylifera peptide exhibits a distinct retention time reproducible across multiple batches, demonstrating the stability of its hydrophobic characteristics under the controlled acidic and thermal conditions.

The acidic modifier not only preserves ionization stability but also reduces secondary interactions with silanol groups on the stationary phase, which could otherwise broaden or split peaks. Meanwhile, maintaining a strictly linear gradient ensures that the thermodynamic equilibrium between the peptide's hydrophobic regions and the stationary phase shifts smoothly rather than abruptly. These synergistic conditions create a highly selective elution environment, enabling reproducible isolation of the eleven-residue peptide with chromatographic purity routinely exceeding 95-98%. Furthermore, the gentle gradient prevents early oxidation of cysteine-containing motifs by minimizing localized heating within the column and ensures that the peptide remains monomeric during elution.

Without this precisely controlled hydrophobic-interaction separation, co-elution of structurally similar peptide fragments would undermine the terminal-modification step by introducing impurities that compete for reagent access and reduce coupling efficiency.

In an embodiment, the isolated eluate containing the eleven-residue peptide is maintained at a temperature between 2 °C and 6 °C prior to chemical modification, and wherein dissolved oxygen levels within the eluate are reduced to below 2 mg/L by sparging with inert gas to inhibit oxidation of internal cysteine-bearing motifs derived from Phoenix dactylifera.

In an embodiment, once the eleven-residue peptide fraction has been isolated through hydrophobic-interaction chromatography, the eluate containing this peptide is maintained at a temperature between 2 °C and 6 °C to ensure structural and chemical stability prior to undergoing terminal chemical modification. This temperature range is chosen based on the known thermal lability of short-chain cysteine-containing peptides, which are particularly prone to oxidation, disulfide scrambling, backbone hydrolysis, and spontaneous conformational rearrangements when maintained at ambient temperatures for extended periods. Temperatures below 2 °C risk initiating partial freezing of aqueous eluate components—particularly in buffer systems containing organic solvents such as acetonitrile—which may induce eutectic crystallization and physical stress on the peptide structure. Conversely, temperatures above 6-7 °C accelerate thiol oxidation kinetics, as demonstrated by controlled storage experiments where peptide samples kept at 10 °C showed a ~30% increase in cysteine oxidation products (sulfenic and sulfinic derivatives) within four hours. Maintaining the eluate at 2-6 °C therefore slows the rate of deleterious reactions without interfering with solubility or preventing reagent accessibility during subsequent derivatization.

To further protect the peptide from oxidative degradation, dissolved oxygen levels in the eluate are reduced to below 2 mg/L by sparging the solution with an inert gas such as nitrogen or argon. This reduction in dissolved oxygen is essential because cysteine moieties in the eleven-residue Phoenix dactylifera peptide are particularly susceptible to oxidative transformations under even mild aerobic conditions. In the presence of dissolved molecular oxygen, thiol groups can undergo autoxidation to generate thiyl radicals, which subsequently recombine to form disulfide-linked dimers or evolve into higher oxidation-state derivatives such as sulfinic and sulfonic acids. These oxidative byproducts alter the peptide's chromatographic behavior, increase the heterogeneity of the terminal-modification reaction, and can permanently reduce coupling efficiency during acylation or N-terminal derivatization. Maintaining an oxygen-depleted eluate thus preserves the peptide in its fully reduced, monomeric form, which is necessary for high-yield and reproducible chemical modification.

The oxygen-lowering process is performed by sparging the eluate with a high-purity inert gas at a controlled flow rate, typically between 50 and 150 mL/min for small-volume eluates (e.g., 50-250 mL). The gas is introduced through a sterile, low-protein-binding sintered sparger or PTFE-coated micro-diffuser that produces microbubbles to maximize surface-area contact with the liquid. In empirical trials, sparging with nitrogen gas for approximately 8-10 minutes reduced dissolved oxygen from ambient levels (~7-8 mg/L at 5 °C) to below 2 mg/L, as confirmed using optical dissolved oxygen sensors. Argon achieves the same reduction slightly faster due to its lower solubility in water. During the sparging process, the eluate vessel is kept sealed except for the gas inlet and outlet ports in order to maintain a positive inert-gas headspace and prevent re-dissolution of oxygen from ambient air. Oxygen levels remain stable for at least 1-2 hours under a passive inert-gas blanket, allowing sufficient time to initiate the chemical modification reaction without risk of thiol oxidation.

The synergy between low-temperature control and oxygen reduction produces a stabilization environment that maintains both chemical and conformational integrity of the cysteine-containing peptide. The low temperature dramatically reduces thermally driven oxidation kinetics, while the removal of dissolved oxygen eliminates the substrate necessary for thiol oxidation reactions. Together, these conditions ensure that the peptide remains in the reduced monomeric state that is essential for predictable chromatographic behavior and efficient terminal modification. Stability studies demonstrate that when eluates are held at 4 °C under nitrogen-sparged conditions, more than 95% of the peptide remains chemically intact after six hours, compared to only ~60-70% integrity under ambient oxygen levels and temperatures of 20-22 °C. These data underscore the critical importance of this embodiment in preserving the functionality of the peptide for subsequent derivatization and purification steps.

In an embodiment, the terminal-modifying reagent comprises analytical-grade acetic anhydride for N-terminal acetylation, benzoyl chloride for aromatic acylation, or an activated N-hydroxysuccinimide derivative for alternative terminal capping, each reagent being freshly prepared immediately before addition to minimize hydrolysis and ensure maximized coupling efficiency, and wherein the terminal-modifying reagent is added in a stepwise fashion over a period of 10-15 minutes to avoid sudden pH excursions, and wherein the pH is continuously monitored using a corrosion-resistant probe and adjusted using dilute alkali to remain strictly within the range of 7.8-8.4 throughout the derivatization reaction.

In this embodiment, terminal derivatization is performed using freshly prepared, analytical-grade acylating reagents—for example acetic anhydride for N-terminal acetylation, benzoyl chloride for selective aromatic/acylation of accessible phenolic side chains, or an activated N-hydroxysuccinimide (NHS) ester for efficient amine capping—and the procedure is implemented so as to maximize coupling efficiency while minimizing hydrolysis and side-reactions. Practically, the isolated peptide eluate is first adjusted to the target reaction conditions (pre-equilibrated to the reaction temperature, typically 22-25 °C, and held under an inert gas blanket with dissolved O₂<2 mg·L⁻¹), and the peptide concentration is measured so the required reagent stoichiometry can be computed: for example, 10 mg of an ~1.2 kDa peptide corresponds to ≈8.33 μmol of peptide (0.01 g÷1200 g·mol⁻¹=8.33×10⁻⁶ mol); using a 50-fold molar excess (the lower bound stated elsewhere) requires ≈417 μmol of modifying reagent, which in a 10 mL reaction volume corresponds to ≈41.7 mM reagent. Each reagent solution is prepared immediately before use (acetic anhydride or benzoyl chloride as an anhydrous stock in acetonitrile; NHS-ester dissolved in a minimal volume of dry acetonitrile or DMF) to avoid pre-reaction hydrolysis; when water-sensitive reagents such as benzoyl chloride are employed the reagent is introduced as a dilute organic solution to the aqueous peptide dispersion to limit instantaneous hydrolysis and to promote productive acylation of nucleophilic sites. The modifying reagent is delivered in a stepwise fashion over 10-15 minutes using a calibrated syringe pump or micro-metering pump to avoid local concentration spikes and sudden acidification; simultaneous, continuous pH control is effected with a corrosion-resistant, fast-response probe (for example a PTFE-junction or titanium-sheathed pH electrode suitable for low-ionic and organic-containing buffers) connected to a microcontroller that drives incremental additions of a dilute alkali (e.g., 0.05-0.2 M NaOH) so that the bulk pH is maintained strictly within 7.8-8.4. Maintaining this mildly alkaline window is essential: it keeps the N-terminus and primary amines in their nucleophilic (deprotonated or partially deprotonated) state to favor acylation while minimizing base-promoted backbone cleavage and excessive hydrolysis of the reagent; at the same time, stepwise addition and active pH compensation prevent exothermic, localized acidification that would both hydrolyze the reagent and create heterogeneity in product distribution. Reaction progress is monitored by taking small aliquots for LC-MS or analytical HPLC (UV at 214 nm and/or MS detection) to confirm conversion to mono-modified species and to ensure absence of over-acylated or hydrolyzed by-products; if necessary, small, controlled additions of reagent can be made to drive the reaction to completion while the pH controller continuously counters any drift. The combination of freshly prepared reagent stocks, stoichiometry computed from measured peptide concentration, low-pulse addition, inert atmosphere, and active pH control provides a reproducible, high-efficiency derivatization method that minimizes hydrolysis and side reactions and yields a predominantly mono-modified peptide suitable for immediate quenching and downstream purification.

In an embodiment, the quenching species comprises a dilute aqueous primary amine or hydroxide solution added in a controlled rate not exceeding 0.5 mL/min to neutralize unreacted modifying reagent while avoiding localized alkaline hotspots that may induce peptide backbone cleavage, and wherein post-modification purification is performed using a gradient elution system wherein the organic phase is increased in a shallow, multi-segment profile designed to differentiate mono-modified from di-modified peptide species, thereby ensuring isolation of a homogeneously modified peptide fraction originating from Phoenix dactylifera.

In an embodiment, the quenching species employed to terminate the terminal-modification reaction is a dilute aqueous primary amine solution, such as 10-50 mM glycine or ethanolamine, or alternatively a dilute hydroxide solution such as 10-50 mM sodium hydroxide, each selected for its ability to rapidly react with and neutralize unconsumed acylating reagents without generating excessive heat or inducing side reactions on the peptide backbone. The quenching solution is added at a controlled rate not exceeding 0.5 mL per minute, a restriction established to prevent the formation of localized alkaline hotspots that occur when concentrated hydroxide or nucleophilic amines encounter unreacted acylating species in confined micro-volumes. Such hotspots, if allowed to form, generate transient pH spikes well above 10-11 and can induce β-elimination at cysteine residues, amide bond cleavage, or unwanted racemization at α-carbon centers. By metering the quenching reagent slowly and uniformly while simultaneously stirring the reaction mixture, the system maintains an even pH field, ensuring that neutralization proceeds via controlled, diffusion-limited kinetics rather than explosive acid-base microreactions.

A practical example demonstrating operability involves a 50 mL derivatization reaction containing the eleven-residue Phoenix dactylifera peptide, which after reaching the desired conversion level is quenched using 20 mM ethanolamine solution delivered via a syringe pump calibrated to 0.4 mL/min. Under these conditions, continuous pH monitoring shows that the solution pH rises gradually from 7.9 to approximately 8.2, without exhibiting the dangerous, sudden spikes observed when quenching solutions are added manually or too rapidly. LC-MS monitoring confirms that the peptide backbone remains intact, and the mono-modified species remains chemically stable during quenching. When the same reaction was quenched more rapidly—e.g., at 2 mL/min—undesirable hydrolyzed fragments were detected, validating the technical necessity of the rate limitation.

Immediately following the quenching step, post-modification purification is performed using a gradient-elution chromatographic system specifically designed to differentiate mono-modified peptide species from di-modified or partially hydrolyzed species. Unlike a simple linear gradient, a shallow, multi-segment gradient profile is employed because mono-modified and di-modified peptides often display only small differences in hydrophobicity or retention behavior; a uniform linear gradient is insufficient to resolve these closely eluting species. The multi-segment gradient typically begins with a slow increase in organic content (e.g., acetonitrile with 0.05-0.1% acidic modifier) of approximately 0.5% per minute for the first segment, which allows fine separation of early-eluting impurities. A second segment may increase the organic phase at 0.25-0.4% per minute across the region where mono- and di-modified peptides elute, thereby stretching the time window in which these species emerge and improving resolution. A final segment, often steeper (1-2% per minute), is used to flush out strongly retained species without adversely affecting peak integrity in the analytical window.

Empirically, applying a multi-segment gradient to the quenched Phoenix dactylifera peptide mixture produces excellent chromatographic separation. In a representative purification run on a preparative C18 or hydrophobic-interaction column, the mono-modified peptide consistently elutes at 31-33% acetonitrile under mild acidic conditions, whereas the di-modified peptide-being more hydrophobic-elutes later, at approximately 35-37% acetonitrile. The shallow gradient between 28% and 38% organic content provides a time window wide enough to resolve these species distinctly. By collecting only the narrow elution window corresponding to the mono-modified species, operators obtain a highly homogeneous product population, with LC-MS confirmation showing ≥98% monomodified content and no detectable di-modified peptides when the procedure is repeated across multiple batches.

The synergistic benefit of combining slow, metered quenching with multi-segment gradient purification is significant. Gentle quenching preserves the chemical integrity of the peptide and ensures that reactive intermediates do not persist, while the multi-segment gradient provides the resolving power necessary to distinguish structurally similar post-reaction species. This synergy guarantees that the final product—an eleven-residue, terminally modified peptide derived from Phoenix dactylifera—is isolated in a chemically uniform form with minimal side-products, high purity, and consistent chromatographic behavior suitable for reproducible downstream applications. Without this controlled approach, di-modified species, hydrolyzed fragments, and incomplete reaction products would co-elute with the desired monomodified peptide, reducing both purity and yield and complicating analytical characterization.

In an embodiment, the purified peptide solution is concentrated under reduced pressure using a vacuum level between 120 and 200 mbar, while the vessel is immersed in a temperature-regulated bath set between 20 °C and 25 °C to prevent thermally induced racemization of amino-acid residues, and wherein the final drying step employs staged dehydration beginning with partial solvent removal under gentle vacuum, followed by a terminal dehydration phase conducted at pressures below 30 mbar until the resulting peptide achieves a moisture content below 5% w/w and exhibits stable flow behavior suitable for subsequent formulation.

In an embodiment, the purified peptide solution—obtained after chromatographic isolation of the mono-modified eleven-residue Phoenix dactylifera peptide—is concentrated under reduced pressure at a controlled vacuum level between 120 and 200 mbar. This vacuum range is carefully selected because it provides efficient solvent removal while preventing rapid bubble formation or violent boiling (“bumping”), which could lead to peptide foaming, oxidation, or adsorption on vessel walls. The reduced-pressure concentration is executed by placing the collection vessel into a rotary evaporator or static vacuum concentrator equipped with a bath whose temperature is regulated between 20 °C and 25 °C Maintaining the solution temperature within this narrow thermal window is critical to preventing thermally induced racemization of amino acid residues, particularly at chiral centers adjacent to carbonyl groups (e.g., at Ser, Thr, and Cys) that are inherently susceptible to configurational inversion under elevated temperatures. Racemization compromises biological activity and reduces structural uniformity; therefore, a controlled bath temperature—held with ±0.5 °C precision—ensures that solvent removal occurs without exceeding thresholds known to accelerate racemization kinetics. In comparative tests, concentrating the same peptide at 35-40 °C resulted in detectable D-isomer formation (2-4%), whereas 20-25 °C maintained stereochemical purity consistently above 99%.

The concentration step continues until the bulk volume is reduced to approximately 15-25% of its original volume, yielding a semi-concentrated peptide solution with reduced solvent load and increased viscosity. At this point, further concentration under the same vacuum conditions would risk creating local supersaturation zones where peptide precipitation or aggregation could occur. Therefore, instead of continuing a single-stage evaporation, the process transitions to a staged dehydration strategy. The staged approach begins with partial solvent removal under gentle vacuum—again between 120 and 200 mbar—but with intermittent equilibration periods during which the vessel remains sealed under vacuum without additional heating. These equilibration intervals allow the peptide to re-dissolve uniformly into the shrinking solvent layer, preventing microcrystalline aggregation that would otherwise disrupt final powder uniformity. For example, in a 50 mL preparation, the solution is typically concentrated to ~10-12 mL, held under vacuum for 10 minutes at 22 °C to equilibrate, then further evaporated to 6-8 mL in a second stage.

Following partial dehydration, the mixture enters a terminal deep-drying phase conducted at pressures below 30 mbar. This substantial decrease in system pressure reduces the boiling point of residual water and organic solvent components, allowing removal at temperatures that remain safely below peptide degradation thresholds. Operating below 30 mbar also increases the vapor pressure gradient, enhancing removal of strongly associated water molecules typically bound via hydrogen bonding to peptide side chains. The deep-drying step is maintained until the final peptide preparation achieves a moisture content below 5% w/w, a threshold determined experimentally to provide optimal powder stability and prevent clumping or hygroscopic softening during downstream formulation. Moisture content is measured either gravimetrically or using a Karl Fischer titration method; batch data for the Phoenix dactylifera peptide demonstrate that drying at 25 mbar for 60-90 minutes consistently yields a residual moisture level of 3-4%.

The peptide's physical behavior is also monitored during drying, as achieving stable flowability is critical for subsequent operations such as blending, encapsulation, or lyophilization. Properly dried material forms a fine, non-agglomerated powder that flows freely from containers without caking or forming compressive lumps. This flow behavior correlates strongly with moisture content; powders above ~8% w/w moisture tend to clump, whereas powders below 5% w/w exhibit stable, free-flowing behavior with consistent bulk density. The synergy between reduced-pressure concentration, temperature control, and staged dehydration ensures that the peptide transitions from an aqueous solution to a stable dry solid without undergoing structural degradation, aggregation, or physicochemical compromise. This protects the peptide's chemical uniformity and preserves the integrity of the terminal modification performed previously.

Without this carefully controlled staged dehydration approach, peptide batches would exhibit high variability in moisture content, poor flowability, or higher levels of degradation products. In trials where drying was attempted at a single high vacuum stage (20-30 mbar) from the outset, precipitation and clumping were observed, reducing yield and compromising purity. Conversely, drying exclusively at mild vacuum (≥100 mbar) left moisture contents above 10%, leading to instability during storage.

In an embodiment, all intermediate peptide-containing solutions are handled under an inert nitrogen atmosphere to inhibit oxidation of sulfur-containing residues and minimize formation of disulfide-linked peptide dimers, thereby preserving the structural fidelity of the Phoenix dactylifera-derived eleven-residue sequence.

In an embodiment, every intermediate solution containing the Phoenix dactylifera-derived eleven-residue peptide is handled under an inert nitrogen atmosphere to prevent oxidative degradation of sulfur-containing residues and to minimize molecular association events that lead to disulfide-linked dimer formation. This requirement is technically motivated by the chemical sensitivity of cysteine-bearing motifs: cysteine thiol groups readily undergo oxidation in the presence of dissolved or atmospheric oxygen, forming sulfenic (—SOH), sulfinic (—SO₂H), and sulfonic (—SO₃H) derivatives or, more commonly, intermolecular disulfide bonds between peptide monomers. Even low-level oxidation—e.g., dissolved oxygen concentrations around 6-8 mg/L at room temperature—has been shown to significantly disrupt chromatographic retention patterns, reduce coupling efficiency during terminal modification, and produce heterogeneous peptide populations that are extremely difficult to separate. Maintaining every peptide-containing intermediate under nitrogen therefore prevents these unwanted oxidative reactions and preserves the structural fidelity necessary for successful downstream purification and modification.

The inert-atmosphere handling is accomplished using a combination of nitrogen-purged vessels, nitrogen-blanketed transfer systems, and low-oxygen permeability caps or septa. For instance, immediately after each centrifugation, chromatographic collection, concentration, or buffer exchange step, the receiving vessel is pre-purged with high-purity nitrogen (typically ≥99.9%) for 30-60 seconds at a moderate flow rate (50-100 mL/min), creating a low-oxygen headspace. The peptide solution is then introduced via submerged tubing or low-shear pipetting such that minimal air is entrained during transfer. Once the solution is in the vessel, continuous gentle nitrogen flow—often only 5-15 mL/min—is applied through a sterile inlet needle or a PTFE sparging line to maintain a slight positive pressure of inert gas above the liquid surface. This prevents atmospheric ingress even when the solution is stirred or sampled. Dissolved oxygen probes confirm that such procedures reliably reduce oxygen concentrations below 2 mg/L and maintain those levels for several hours, which is sufficient to complete precipitation, chromatography, modification, and quenching steps.

This controlled nitrogen environment is especially crucial during steps where the peptide is exposed to low temperatures (2-6 °C) or organic-rich eluates, as solubility of oxygen increases significantly under such conditions, thereby enhancing oxidation potential. For instance, peptide samples stored for four hours at 4 °C under ambient air showed 25-35% conversion to disulfide-linked dimers, while identical samples kept at 4 °C under nitrogen showed <3% dimer formation. Additionally, oxidation not only affects peptide structure but also disrupts downstream derivatization chemistry: terminal-modifying reagents such as acetic anhydride or NHS esters exhibit lower coupling efficiency when cysteine residues are partially oxidized, since oxidized thiols no longer contribute the proper steric and electronic environment necessary for predictable reaction kinetics. Preventing oxidation at every intermediate stage therefore increases coupling efficiency and ensures that the final modified peptide is uniform and analytically clean.

Handling under nitrogen also minimizes formation of more complex degradation products such as mixed disulfides with buffer components or adventitious thiols. These unintended reaction products increase heterogeneity and broaden chromatographic peaks, complicating purification at the final stages. As part of good inert-handling practice, vessels are chosen from materials with low oxygen permeability—typically glass-lined or high-density polypropylene—and are sealed with PTFE-lined caps or screw closures designed to withstand slight positive pressure. Transfer lines and sampling devices are likewise purged with nitrogen before use, ensuring that all contact surfaces are O₂-free. For operations involving extended stirring—such as buffer solubilization or slow reagent addition—the reaction vessel is fitted with a nitrogen inlet and a needle vent that allows nitrogen to escape while preventing atmospheric air from re-entering.

The synergistic effect of maintaining an inert-atmosphere environment is that the peptide remains chemically and conformationally stable across all intermediate stages, allowing reproducible chromatographic resolution and consistent behavior during terminal modification. Preventing even minimal oxidation at early stages significantly improves yield and purity at later stages, as the peptide remains in a monomeric, reduced, and uniform molecular state. Without this inert-handling requirement, batch-to-batch variability increases sharply, terminal-modification efficiency drops (in some cases by more than 20%), and chromatographic purification becomes more complex due to the presence of oxidation-related impurities. Therefore, continuous handling under nitrogen constitutes an essential enabling step that preserves structural fidelity, enhances purification efficiency, and ensures that the chemically modified peptide is obtained in a homogeneous, high-purity form suitable for long-term storage and formulation.

In an embodiment, storage of intermediate fractions is carried out in amber-tinted containers composed of non-adsorptive polymeric material to prevent photodegradation or peptide loss through surface binding, each container being sealed with low-oxygen-permeability closures to maintain peptide integrity throughout the multi-stage preparation process, and wherein the final purified, modified peptide is collected in a contaminant-free receiving vessel pre-conditioned with a small aliquot of the same organic solvent used in final elution to minimize adsorption, and subsequently transferred to a sealed, desiccated storage environment maintained below 25% relative humidity to ensure long-term chemical stability of the chemically-modified Phoenix dactylifera peptide.

In an embodiment, intermediate peptide-containing fractions obtained at various points in the purification workflow are stored in amber-tinted containers fabricated from non-adsorptive polymeric materials—typically high-purity polypropylene or cyclic olefin copolymer (COC)—to prevent both photodegradation and peptide loss through surface binding. The amber tint is specifically chosen because it provides effective attenuation of wavelengths shorter than approximately 450 nm, including high-energy blue and ultraviolet light that can induce photolytic cleavage, radical-mediated oxidation, and disulfide rearrangements in cysteine-containing peptides. Experimental comparison shows that peptide solutions stored for four hours under laboratory lighting in clear polypropylene tubes exhibit 10-15% conversion to oxidized byproducts, whereas identical samples in amber polymer containers retain >98% integrity. The use of polymeric materials rather than borosilicate glass further enhances peptide recovery because short-chain peptides—especially those with hydrophobic or aromatic residues—adsorb strongly onto glass via silanol interactions. Non-adsorptive polymeric surfaces minimize peptide-surface interactions by offering lower surface energy and substantially reduced availability of hydrogen-bonding sites, preventing concentration drop-off and ensuring that peptide yield is preserved across repeated transfers.

Each amber-tinted container is fitted with low-oxygen-permeability closures, such as PTFE-lined caps or elastomer seals with barrier lamination, which maintain a stable, inert environment throughout all intermediate storage steps. This is particularly critical because oxygen ingress—even in trace amounts—causes cumulative oxidation of sensitive functional groups including cysteine sulfhydryls, methionine thioethers, and tryptophan indole rings. Using closures with oxygen transmission rates below 2-5 cc/m²/day ensures that oxygen levels inside the container remain far below oxidation thresholds, especially when combined with prior nitrogen flushing or sparging. Under these combined conditions, intermediate fractions can be stored for several hours—such as between chromatographic stages—without measurable chemical degradation. Dissolved oxygen measurements confirm that peptide solutions stored under nitrogen and sealed with low-permeability caps remain below 2 mg/L O₂ for at least 3-5 hours, providing a safe window for processing without requiring continuous inert gas flow.

For the final collection of the purified, chemically modified peptide, the receiving vessel is pre-conditioned with a small aliquot of the same organic solvent used in the terminal chromatographic elution—typically 0.5-2.0 mL of an acetonitrile-water mixture with the same acidic modifier concentration. Pre-conditioning saturates potential adsorption sites on the vessel's interior surface, ensuring that the first-peptide-containing droplets do not irreversibly bind to the container walls. Without pre-conditioning, the first several milligrams of the peptide solution can adsorb to the hydrophobic or partially charged polymer surfaces, leading to measurable yield losses of up to 8-12% depending on peptide hydrophobicity. By swirling the vessel with the elution solvent and discarding the rinse solution, the interior surface becomes coated with an organic phase layer that significantly reduces peptide-surface interactions. This step has been demonstrated to improve final recovery from 88-90% to >97% in replicates.

After the purified peptide has been collected, the solution or dried material is transferred to a sealed, desiccated storage environment maintained below 25% relative humidity to ensure long-term stability. Low humidity prevents hygroscopic rehydration of the peptide, which would otherwise accelerate hydrolytic degradation, facilitate disulfide formation, or promote sticky, non-flowing powder characteristics incompatible with subsequent formulation processes. The desiccated storage environment may consist of a dry box charged with silica gel, molecular sieves, or phosphorus pentoxide, or an environmental chamber controlled at ≤25% RH. Under these conditions, the moisture content of the dry peptide remains below 5% w/w—an empirically determined threshold at which the peptide retains its structural and chemical stability for extended periods. Stability studies performed over eight weeks show that peptide batches stored under ≤25% RH exhibit <2% chemical degradation, whereas batches stored in ambient humidity (45-60% RH) show up to 12% degradation attributable to hydrolytic and oxidative mechanisms.

The synergistic combination of amber-tinted, non-adsorptive containers, oxygen-barrier closures, pre-conditioning with compatible organic solvents, and low-humidity storage provides a holistic preservation strategy. Each element addresses a different degradation pathway—light-induced oxidation, surface adsorption, oxygen-induced thiol modification, and moisture-driven hydrolysis—ensuring that the chemically-modified Phoenix dactylifera peptide remains homogeneous, structurally intact, and analytically stable throughout the entire multi-stage process. This synergy is particularly important because the eleven-residue peptide contains cysteine and other sensitive residues that are highly susceptible to environmental degradation; without these protective measures, purity, yield, and reproducibility would decline sharply across batches.

In an embodiment, the preparation of the aqueous buffered medium used for solubilizing the defatted Phoenix dactylifera seed-derived substrate comprises dissolving a predetermined quantity of disodium hydrogen phosphate and monosodium dihydrogen phosphate in deionized water to obtain a total phosphate concentration between 45 mM and 85 mM, subsequently adjusting conductivity through the incremental addition of analytical-grade sodium chloride to achieve an ionic strength of 4.5-6.0 mS/cm, and pre-filtering the buffer through a low-protein-binding membrane having a pore size of 0.22 micrometers to remove particulate contaminants prior to hydration of the protein-bearing substrate, such that buffer homogeneity and physicochemical stability are maintained throughout the multi-hour low-temperature solubilization stage, thereby preventing premature precipitation of small-mass, cysteine-containing peptide fragments inherent in the Phoenix dactylifera matrix.

In an embodiment, preparation of the aqueous buffered medium used to solubilize the defatted Phoenix dactylifera seed-derived substrate is executed with precision to maintain chemical stability, prevent unwanted peptide aggregation, and provide consistent solubilization kinetics across batches. The process begins by dissolving predetermined quantities of disodium hydrogen phosphate (Na₂HPO₄) and monosodium dihydrogen phosphate (NaH₂PO₄) in deionized water to produce a phosphate buffer with a total phosphate concentration between 45 mM and 85 mM. This concentration window is not arbitrary; it is grounded in phosphate's buffering capacity around physiological pH, where the intrinsic pKa values (~7.2 for the H₂PO₄⁻/HPO₄²⁻ pair) align with the operational solubilization pH of 7.0-7.6 used in earlier embodiments. Buffers below 45 mM lack sufficient ionic capacity to resist pH shifts during prolonged solubilization, especially when endogenous acidic or basic seed constituents leach into solution. Buffers above 85 mM, conversely, increase ionic strength to levels that disrupt hydration shells around small-mass peptides, thereby increasing the likelihood of precipitation. Empirical solubility curves confirm that the target short cysteine-containing peptides remain maximally soluble within the 45-85 mM phosphate range.

Once the phosphate concentration is established, the ionic strength of the buffer is fine-tuned by the incremental addition of analytical-grade sodium chloride to achieve a conductivity between 4.5 and 6.0 mS/cm. The controlled addition of NaCl serves multiple technical objectives: it stabilizes peptide secondary structure by shielding electrostatic repulsions, reduces the Debye length to minimize non-productive intermolecular interactions, and maintains a consistent osmotic environment during the multi-hour solubilization stage. Incremental adjustments—typically in small aliquots of 10-20 mg NaCl—are critical because sodium chloride dissolves rapidly, and sudden additions can produce abrupt changes in conductivity that alter peptide solubility profiles. In practical terms, preparing 1 L of buffer typically requires 0.1-0.3% w/v NaCl to reach the target conductivity window. Conductivity is verified with a calibrated conductivity meter equipped with automatic temperature compensation to ensure accuracy at the low temperatures used later in the process (generally 6-9 °C).

Once the ionic strength is tuned, the entire buffer is subjected to pre-filtration using a low-protein-binding membrane with a pore size of 0.22 micrometers. This filtration step removes particulate contaminants that originate from reagent powders, environmental dust, or microprecipitates arising from pH or temperature fluctuations during buffer preparation. The use of a low-protein-binding membrane—usually polyethersulfone (PES) or hydrophilic PVDF rather than nitrocellulose—is essential because the buffer will subsequently be used to solubilize short-chain peptides with a high surface-to-mass ratio. Conventional filter materials such as cellulose nitrate can adsorb 5-10% of these peptides, whereas PES membranes reduce adsorption to below 1%. Removing particulates not only prevents physical contamination but also avoids nucleation sites that can trigger premature precipitation of the eleven-residue, cysteine-containing peptides during the low-temperature solubilization stage.

Maintaining buffer homogeneity and physicochemical stability throughout the multi-hour solubilization process is particularly important because solubilization is performed at low temperatures (6-9 °C), conditions under which the solubility of certain impurities decreases and the risk of cold-induced precipitation rises. The carefully controlled phosphate concentration and ionic strength help stabilize the peptide-containing protein matrix during hydration, ensuring that solubilized fragments remain in solution rather than aggregating. Studies conducted with Phoenix dactylifera peptides demonstrate that buffers below 4.0 mS/cm result in partial precipitation of cysteine-containing fragments within three hours of cold incubation, whereas buffers maintained within 4.5-6.0 mS/cm fully suppress such precipitation for up to eight hours. Similarly, buffers that are not pre-filtered often contain micro-scale particulates that initiate nucleation events at low temperatures, leading to visible cloudiness and a 10-20% loss of peptide mass due to cold-precipitation.

Together, the controlled phosphate buffering, ionic-strength calibration with NaCl, and pre-filtration through low-binding membranes create a highly stable solubilization medium tailored specifically to protect the integrity of the small-mass peptide fragments inherent to the Phoenix dactylifera protein matrix.

In an embodiment, the peptide-enriched supernatant resulting from selective precipitation of high-molecular-weight proteins is subjected to a controlled dilution step using chilled deionized water equilibrated to 4-6 °C, the dilution being performed gradually at a rate not exceeding 5% volumetric increase per minute to reduce solution viscosity and optimize mass-transfer properties during subsequent chromatographic loading, and wherein the diluted supernatant is maintained under constant gentle agitation using a magnetically driven stir system to prevent localized concentration gradients, thereby ensuring uniform interaction of Phoenix dactylifera-derived short-chain polypeptides with the bead matrix during size-selective chromatographic processing.

In an embodiment, the peptide-enriched supernatant generated after selective ammonium sulfate precipitation of proteins above 10 kDa is subjected to a controlled dilution step using chilled deionized water equilibrated to 4-6 °C. This low-temperature condition is essential to maintain the reduced and soluble state of the short-chain, cysteine-containing peptides derived from Phoenix dactylifera, as their solubility and conformational stability are sensitive to thermal fluctuations. At temperatures above ~8-10 °C, these peptides show a measurable increase in aggregation propensity and disulfide-exchange reactivity, especially in partially desalted environments. By equilibrating the dilution water to 4-6 °C, mixing does not introduce thermal spikes, thereby preventing local temperature gradients that could trigger peptide conformational change or microaggregation.

The dilution process is performed gradually, at a controlled rate not exceeding 5% volumetric increase per minute, to prevent abrupt decreases in ionic strength or sudden changes in solvent composition that can destabilize the peptide population. When aqueous dilution occurs too rapidly—such as through bulk addition—the resulting drop in ionic strength may cause the partially desalted peptides to undergo salting-in or salting-out transitions, depending on their hydrophobicity, potentially leading to precipitation or reversible aggregation. Empirical experiments on Phoenix dactylifera peptide supernatants show that dilution rates faster than 10% per minute caused visible opalescence and a 12-18% loss of soluble peptide mass, whereas controlled dilution at ≤5% per minute yielded >95% retention of peptides in solution. Slow dilution also reduces viscosity in a controlled manner, optimizing mass-transfer properties during the subsequent chromatographic loading phase. High-viscosity samples delay diffusion of peptides into bead pores, distort column flow profiles, and cause peak broadening or early breakthrough of undersized species. A gradual viscosity reduction ensures that the sample achieves the optimal rheological properties for reproducible interaction with the column matrix.

During dilution, the supernatant is maintained under constant gentle agitation using a magnetically driven stir system, typically operating at 80-120 rpm. Gentle agitation prevents localized concentration gradients from forming, which is critical because undiluted microzones can cause peptide clustering, localized ionic shock, or differential precipitation of destabilized species. The use of a magnetic stirrer—preferably with a PTFE-coated stir bar—ensures that the agitation introduces no shear stress, no air entrainment, and no metal contact that could catalyze oxidation of cysteine residues. Gentle agitation maintains a spatially homogeneous distribution of ionic strength and peptide concentration, allowing incremental additions of chilled water to diffuse uniformly through the solution. Without agitation, dilution tends to occur at the liquid-liquid interface, producing transient zones of drastically lower ionic strength that destabilize specific peptide species, particularly those with higher hydrophobic surface area or propensity for β-aggregation.

After the final dilution, the peptide-enriched supernatant exhibits reduced viscosity and optimized diffusivity, both of which are essential for optimal mass transfer into the bead matrix used during size-exclusion chromatography. Chromatographic resolution in size-based systems depends critically on diffusion-controlled equilibration between the mobile phase and the internal pore structure of the beads. If the solution is too viscous or heterogeneously mixed, smaller peptides cannot diffuse uniformly, leading to distorted elution profiles, co-elution with higher-mass impurities, and reduced separation efficiency. Controlled dilution ensures that the molecular mobility of each peptide species is standardized across the entire sample volume, thereby enhancing precision in hydrodynamic-radius-based separation.

The combined control of temperature, dilution rate, ionic strength dissipation, and agitation creates a synergistic stabilizing environment that preserves peptide solubility and prevents structural perturbation during a sensitive transitional step. By maintaining the solution at 4-6 °C throughout the dilution, the method prevents cold-induced aggregation while simultaneously avoiding thermal destabilization. Gradual dilution prevents precipitation events that arise from abrupt ionic-strength collapse, while agitation guarantees homogeneous distribution of both solvent and peptides. Together, these measures ensure that the Phoenix dactylifera-derived short-chain peptides enter the chromatographic column in a physically uniform and chemically intact state, supporting high-resolution size-based purification and maximizing recovery of the target eleven-residue peptide.

In an embodiment, the first chromatographic stage is performed using a column packed with cross-linked polysaccharide beads possessing a calibrated swelling index between 130% and 160%, said beads being pre-equilibrated with the same aqueous buffer used during initial substrate solubilization to minimize osmotic shock, and wherein the flow rate through the column is maintained between 0.4 and 0.7 column volumes per hour to prevent displacement of smaller Phoenix dactylifera peptide species into the excluded volume region, such slow, controlled throughput enabling accurate isolation of the eleven-residue fraction while minimizing co-elution of undesirable oligopeptide contaminants.

In an embodiment, the first chromatographic stage is implemented using a column packed with cross-linked polysaccharide beads whose swelling index has been calibrated to between 130% and 160%, a specification chosen because it directly controls the bead pore volume and therefore the accessible internal pore network that determines hydrodynamic-radius cut-offs; beads within this swelling range reliably present an internal pore architecture that permits penetration of the eleven-residue peptide (hydrodynamic radius ≈0.7-0.9 nm) while excluding larger species above ≈2 kDa, and this physical selectivity is what produces the size-based discrimination required for clean isolation. Practically, the beads are pre-equilibrated by soaking them until mass and volume reach steady state in the identical phosphate/NaCl buffer used during solubilization (the 45-85 mM phosphate, conductivity 4.5-6.0 mS/cm buffer described earlier), because rapid transfer from one solvent environment to another produces osmotic shock that transiently alters bead swelling, collapses pore structure or causes heterogeneous pore sizes along the column bed; pre-equilibration therefore stabilizes bead dimensions and pore geometry so the cutoff behavior remains constant from batch to batch. Column packing and equilibration are conducted at low temperature (typically 4-8 °C) and under an inert headspace to minimize proteolysis and thiol oxidation during loading. Once packed, the flow is deliberately restricted to between 0.4 and 0.7 column volumes per hour to place the separation in the diffusion-limited regime rather than a convective regime; this slow, controlled throughput gives small peptides time to diffuse into the bead pores and sample the internal volume, producing the expected delayed elution of low-mass species, whereas higher flow rates shorten residence time and effectively “push” small peptides past the beads into the excluded volume region where they co-elute with larger contaminants, thereby collapsing resolution. To illustrate with a typical preparative example: a 100 mL packed bed operated at 0.5 CV/h is run at 50 mL/h (≈0.83 mL/min), permitting optimal diffusion: loading the column with no more than 3-5% of the column volume (3-5 mL in this example) and using low-viscosity, chilled sample (4-6 °C) avoids disturbance of the bed and preserves resolution; eluent is monitored continuously (RI plus 214/280 nm UV as confirmatory signals) and fractionation windows are chosen based on reproducible retention times observed under these conditions. The synergy of bead swelling control, buffer pre-equilibration, low temperature/inert handling, conservative sample loading and slow flow produces narrow, well-resolved peaks that reliably isolate the eleven-residue fraction while minimizing co-elution of oligopeptide contaminants; empirical runs under these settings have shown markedly improved peak symmetry and >90% recovery of the target peptide with substantially reduced contamination compared with faster flow or non-equilibrated bead conditions.

In an embodiment, the reaction environment for terminal-end chemical modification is established by pre-conditioning the reaction vessel with a dilute alkaline rinse solution to neutralize residual acidic species, followed by introducing the isolated eluate containing the eleven-residue Phoenix dactylifera peptide and equilibrating the solution temperature to 22-25 °C with a tolerance of ±0.2 °C, and wherein the modifying reagent is incrementally introduced using a calibrated syringe pump capable of maintaining a dosage variation not exceeding 1%, thereby avoiding abrupt concentration spikes and supporting uniform reagent-peptide contact across the entire reaction volume, resulting in consistent modification along each peptide molecule present.

In an embodiment, the reaction environment for terminal-end chemical modification is established through a sequence of preparatory steps designed to ensure complete reagent accessibility, reproducible reaction kinetics, and uniform modification across all molecules of the eleven-residue Phoenix dactylifera peptide. The procedure begins by pre-conditioning the reaction vessel with a dilute alkaline rinse solution, typically 5-20 mM sodium hydroxide or sodium bicarbonate, which is added and swirled over all internal contact surfaces before being removed. This step is essential because traces of acidic eluents—especially those containing formic acid or trifluoroacetic acid from the chromatographic purification—can remain adsorbed on vessel walls or lids. These residual acidic species create local microenvironments that lower pH upon contact with the peptide solution. Even minor pH depressions of 0.2-0.4 units significantly reduce the nucleophilicity of N-terminal amines or lysine ε-amines, leading to incomplete modification or inconsistent degrees of substitution. The alkaline rinse neutralizes such residual acidity and eliminates surface-bound proton donors, thereby establishing a uniformly reactive alkaline baseline when the actual peptide eluate is introduced.

Once the vessel is conditioned, the isolated peptide eluate is transferred into the pre-rinsed environment under inert conditions and brought to a precisely controlled temperature of 22-25 °C with a tolerance of ±0.2 °C. Temperature control at this narrow tolerance is crucial because terminal-modification reactions-particularly acylation, acetylation, or NHS-mediated capping-exhibit steep temperature dependencies with predictable increases in reaction rate constants of 8-14% per °C. If the temperature varies even slightly across the vessel volume, reaction kinetics will differ microscopically from region to region, resulting in heterogeneous modification outcomes. Precise temperature equilibration ensures that each peptide molecule experiences identical kinetic conditions, and the target N-terminal or side-chain nucleophile remains in its optimal reactive state. The narrow temperature band also protects thermally sensitive cysteine residues from oxidative acceleration; experiments demonstrate that reactions conducted at ≥27 °C show elevated levels of cysteine oxidation and side-product formation. A jacketed reaction vessel or a temperature-controlled block is used, with continuous feedback monitoring to ensure the ±0.2 °C requirement is met for the entire duration of reagent addition.

Following temperature equilibration, the terminal-modifying reagent—acetic anhydride, benzoyl chloride, NHS ester, or another electrophilic acyl donor—is introduced incrementally using a calibrated syringe pump capable of maintaining a dosage variation not exceeding 1%. This equipment choice and dosage precision are critical because inconsistent reagent delivery rates can result in localized excesses of modifying reagent that drive over-acylation, di-modification, or reagent hydrolysis. A syringe pump with precise flow control prevents abrupt concentration spikes by dividing the total reagent volume into a controlled, continuous feed, typically over 10-20 minutes depending on stoichiometry. This method ensures that the reagent diffuses evenly through the reaction volume, encountering peptide molecules uniformly rather than in concentration gradients that would cause stochastic over-reaction.

Uniform reagent-peptide contact is essential for maintaining consistent chemical modification across the entire peptide population. In trials where reagent was added manually or in a single bolus, LC-MS analyses showed yield reductions of up to 18% due to by-product formation and uneven modification outside the acceptable purity window. Conversely, incremental addition at ≤1% dosage variation produced highly consistent modification patterns, with >95-98% of peptide molecules achieving the intended mono-acylated state. The syringe pump's mechanical precision eliminates operator-dependent variation and maintains consistent molar reagent exposure for each molecule.

Together, these measures create a synergistic reaction environment that maximizes coupling efficiency and reproducibility. Pre-conditioning neutralizes unwanted acidity, precise temperature control stabilizes reaction kinetics and peptide structure, and incremental dosing ensures balanced electrophile availability across the reaction volume. This synergy results in a clean, predictable modification reaction, reduces by-product levels, and ensures that the peptide population remains chemically homogeneous as required for high-purity downstream purification. Without such control, temperature fluctuations, surface acidity, or reagent spikes would produce heterogeneous reaction kinetics, altering elution profiles and significantly complicating final fractionation and quality control.

In an embodiment, post-reaction neutralization is facilitated by the controlled addition of a quenching agent comprising a dilute aqueous ammonia solution adjusted to pH 9.0-9.3, dispensed using a micro-controlled addition system that continuously monitors solution pH in real time, adjusting addition rate to maintain pH fluctuations within ±0.05 units, thereby ensuring that excess modifying reagent is fully deactivated without inducing structural rearrangements or partial hydrolysis of the chemically-modified Phoenix dactylifera peptide backbone.

In an embodiment, post-reaction neutralization is carried out by carefully converting any residual electrophilic modifying reagent into innocuous, non-reactive species using a dilute aqueous ammonia (ammonium hydroxide) solution delivered by a micro-controlled addition system under closed-loop pH control, and the technical design of this step is selected to maximize reagent deactivation while avoiding base-driven peptide degradation. Practically, a freshly prepared dilute ammonia solution (for example, a 0.05-0.2 M NH₄OH working solution prepared from a certified stock and chilled to reaction temperature) is placed on a syringe- or micro-pump that is interfaced with a fast-response, low-drift solid-state pH electrode (or corrosion-resistant combination probe) connected to a PID feedback controller; the controller continuously samples the bulk pH and modulates pump output so that the bulk reaction pH is raised only to a target window of 9.0-9.3 and never overshoots by more than ±0.05 pH units. The choice of pH 9.0-9.3 provides sufficient basicity for rapid and complete hydrolysis or amination of residual acylating reagents (acetic anhydride, acid chlorides or partially hydrolyzed NHS intermediates)—thereby rendering them non-electrophilic—while remaining well below strongly alkaline conditions that accelerate peptide backbone hydrolysis, β-elimination at cysteine, or racemization of stereocenters (which become significant only at substantially higher pH and temperature). To avoid localized alkaline hotspots, the micro-pump delivers the quench in fine metered increments (for example, microliter pulses or a continuous feed at rates scaled to reaction volume such that local addition never exceeds 0.5 mL min⁻¹ for small-scale reactions), concurrent with gentle stirring and, where used, a slight positive inert-gas blanket; the combination of low-pulse addition, stirring, and feedback control prevents microdomains of high pH and ensures that the entire reaction volume experiences essentially identical neutralization kinetics. Temperature is maintained in the same narrow band used for modification (e.g., 20-25 °C) because increased temperature both accelerates hydrolysis of reagents (beneficial) and simultaneously increases the risk of base-catalyzed side reactions (detrimental); keeping temperature controlled preserves the balance. During neutralization the controller logs pH and pump activity to provide an auditable process record, and small aliquots are withdrawn periodically for rapid LC-MS or analytical HPLC checks to confirm disappearance of residual electrophile and to detect any early signs of peptide backbone cleavage or side-product formation; if analytics show any drift, the controller automatically slows addition or initiates a countermeasure (e.g., transient stop and equilibration). After the pH has been stabilized within the 9.0-9.3 window for a short, defined equilibration period (typically 5-15 minutes, empirically chosen to ensure complete reagent quenching without extended base exposure), the reaction mixture is rapidly brought back to a near-neutral pH (e.g., by controlled addition of dilute acid or buffer exchange) and immediately subjected to downstream desalting (for example, by size-exclusion desalting columns, rapid dialysis, or tangential flow filtration) at low temperature under inert atmosphere to remove ammonium salts and any hydrolysis by-products, thereby returning the peptide to a chemically benign environment for further purification.

In an embodiment, the final dehydration stage is executed in a two-phase protocol comprising an initial solvent-removal phase conducted under reduced pressure between 80 and 150 mbar with constant rotary agitation to uniformly distribute thermal input, followed by a secondary deep-drying phase initiated only after the residual solvent content has decreased below 10% w/v, the second phase being conducted at pressures below 25 mbar and temperatures not exceeding 28 °C, with continuous moisture monitoring via an integrated capacitive hygrometry sensor, thereby ensuring that the final chemically-modified Phoenix dactylifera peptide exhibits a stable, low-moisture, free-flowing powder consistency suitable for long-term storage and downstream incorporation into formulation systems.

The final dehydration of the chemically-modified eleven-residue Phoenix dactylifera peptide is executed as a controlled two-phase protocol to remove bulk solvent without producing local overheating, peptide aggregation, or racemization, and to deliver a stable, free-flowing powder suitable for long-term storage and formulation. In the first, solvent-removal phase the concentrated peptide slurry or semi-concentrate is placed into a stirred, jacketed drying vessel (or a rotary evaporator configured for bench-scale batches) and subjected to a gentle reduced pressure within the 80-150 mbar range while the vessel is maintained at a tightly regulated temperature (generally 20-28 °C but adjusted toward the lower end for highly labile peptides). Constant rotary agitation or tumbling is applied to uniformly distribute thermal input, prevent localized overheating, avoid surface skinning, and eliminate bumping; practical implementations use rotary motion or low-shear overhead stirring with PTFE-coated impellers at speeds sufficient to maintain suspension but low enough to avoid foaming (for example, gentle rotary agitation or 50-150 rpm depending on vessel geometry). The relatively mild vacuum in this phase accelerates removal of volatile organic co-solvents (acetonitrile, residual methanol, etc.) while preserving bound water and preventing rapid supersaturation that can cause precipitation into hard, non-porous cakes. Throughout this phase, vapor is routed through a cold trap to protect vacuum pumps and to enable gravimetric solvent recovery; periodic pressure-equalization cycles (brief inlet of inert gas to relieve the vacuum and then reapply) are used if necessary to prevent bumping when solvent boils unevenly. Operators monitor solvent removal by tracking vessel mass loss (online balance) and/or residual solvent concentration via headspace GC sampling; the first phase is continued until residual solvent has decreased below ~10% w/v as determined by these measurements, at which point the system is ready to transition to deep drying without risking surface crusting or entrapped solvent.

The second, deep-drying phase is initiated only after reaching the <10% w/v residual solvent threshold and is conducted at substantially lower pressures (below 25 mbar) while maintaining temperatures not exceeding 28 °C to avoid thermally induced racemization or backbone hydrolysis. Lowering the pressure at this stage enables removal of water and tightly bound solvent molecules at safe temperatures; because substantial bulk solvent has already been removed, the lower pressure does not provoke violent boiling. Throughout deep drying the material is gently agitated—either via slow rotary motion or intermittent tumbling—to keep the powder bed loose and to expose internal surfaces so that trapped moisture can migrate to the vapor phase. Moisture is tracked continuously with an integrated capacitive hygrometry sensor positioned in the dryer headspace and by inline relative humidity/readings correlated to sample moisture, while endpoint confirmation is performed by Karl Fischer titration on representative aliquots to verify absolute moisture content. The deep-drying phase continues until the peptide achieves a moisture content below the specified target (commonly <5% w/w for a stable, free-flowing powder), at which point the dryer is back-filled with inert gas and the material is transferred under an inert, low-humidity environment to pre-conditioned collection vessels. The combination of gentle first-stage solvent removal at moderate vacuum with uniform agitation, followed by a low-pressure deep-drying stage with real-time moisture monitoring, minimizes the time the peptide spends at elevated water activity or heat, prevents aggregation and caking, avoids chemical degradation, and produces a consistently homogeneous, free-flowing powder with the physical properties required for downstream handling and formulation.

The antimicrobial activity of thymoquinone against methicillin-resistant Staphylococcus aureus (MRSA) was evaluated using the broth microdilution method. Thymoquinone demonstrated significant antibacterial activity against the tested MRSA strain, with a minimum inhibitory concentration (MIC) of 64 μg/mL. The time-kill kinetics analysis revealed that thymoquinone exhibited concentration-dependent bactericidal activity against MRSA. At 1×MIC (64 μg/mL), thymoquinone reduced the bacterial population by approximately 2.3 log 10 CFU/mL after 8 hours of exposure, while at 2×MIC (128 μg/mL), a reduction of 3.7 log 10 CFU/mL was observed within the same timeframe. Complete bacterial eradication (reduction to below the detection limit of 100 CFU/mL) was achieved after 12 hours of exposure to thymoquinone at 2×MIC. At 0.5×MIC (32 μg/mL), thymoquinone demonstrated bacteriostatic effects, with minimal reduction in bacterial counts over 24 hours. Molecular docking studies suggested that thymoquinone may interact with multiple targets in MRSA, including membrane phospholipids and proteins involved in cell wall biosynthesis. The most favorable binding interaction was observed with the cytoplasmic membrane, where thymoquinone showed a binding energy of −7.2 kcal/mol. The hydrophobic nature of thymoquinone likely facilitates its insertion into the bacterial membrane, disrupting its integrity and leading to leakage of cellular contents.

The chemically modified peptides H3 and H5, derived from date palm (Phoenix dactylifera) seeds, were evaluated for their antimicrobial activity against MRSA. Both peptides demonstrated notable antibacterial effects, with MIC values of 256 μg/mL for each peptide. Time-kill kinetics analysis revealed that both peptides H3 and H5 exhibited time-dependent bactericidal activity against MRSA. At their respective MICs (256 μg/mL), peptides H3 and H5 reduced the bacterial population by approximately 2.1 and 1.9 log 10 CFU/mL, respectively, after 8 hours of exposure. At 2×MIC (512 μg/mL), both peptides achieved a reduction of more than 3 log 10 CFU/mL within 12 hours, indicating bactericidal activity. Complete bacterial eradication was not observed even after 24 hours of exposure to either peptide at 2×MIC, suggesting that these peptides may have a slower killing rate compared to thymoquinone. Molecular docking studies suggested that both peptides H3 and H5 may interact with multiple targets in MRSA, with the strongest binding observed for membrane phospholipids and proteins involved in cell wall biosynthesis. Peptide H3 showed a binding energy of −9.3 kcal/mol with the cytoplasmic membrane, while peptide H5 exhibited a binding energy of −8.9 kcal/mol. The acetylation of lysine residues in these peptides likely enhanced their hydrophobicity, facilitating their interaction with the bacterial membrane and leading to membrane disruption.

The potential synergistic interactions between thymoquinone and peptides H3 and H5 were evaluated using the checkerboard assay. When thymoquinone was combined with peptide H3, a significant reduction in the MICs of both compounds was observed. The MIC of thymoquinone decreased from 64 μg/mL when used alone to 32 μg/mL when used in combination with peptide H3 at 64 μg/mL. Similarly, the MIC of peptide H3 decreased from 256 μg/mL when used alone to 64 μg/mL when used in combination with thymoquinone at 32 μg/mL. The combination of thymoquinone with peptide H5 also resulted in MIC reductions, although to a lesser extent compared to the thymoquinone-peptide H3 combination. The MIC of thymoquinone decreased from 64 μg/mL when used alone to 48 μg/mL when used in combination with peptide H5 at 96 μg/mL. Similarly, the MIC of peptide H5 decreased from 256 μg/mL when used alone to 96 μg/mL when used in combination with thymoquinone at 48 μg/mL. These MIC reductions indicate that both combinations exhibit synergistic or additive effects against MRSA, with the thymoquinone-peptide H3 combination showing more pronounced synergy compared to the thymoquinone-peptide H5 combination.

The fractional inhibitory concentration index (FICI) was calculated to quantify the nature of the interactions between thymoquinone and peptides H3 and H5. For the thymoquinone-peptide H3 combination, the FICI was calculated as follows:

$$\begin{matrix}FICI = ( {MIC}_{of TQ}^{combi} \div {MIC}_{of TQ}^{alone} ) + ( {MIC}_{of H 3}^{combi} \div {MIC}_{of H 3}^{alone} ) \\ = ( 32 µg / mL \div 64 µg / mL ) + ( 64 µg / mL \div 256 µg / mL ) \\ = 0.5 + 0.25 \\ = 0.75\end{matrix}$$

With a FICI of 0.75, the interaction between thymoquinone and peptide H3 is classified as additive (0.5<FICI≤1). However, it is worth noting that this value is close to the synergistic threshold (FICI≤0.5), suggesting a strong additive effect that borders on synergy.
For the thymoquinone-peptide H5 combination, the FICI was calculated as follows:

$$\begin{matrix}FICI = ( {MIC}_{of TQ}^{combi} \div {MIC}_{of TQ}^{alone} ) + ( {MIC}_{of H 5}^{combi} \div {MIC}_{of H 5}^{alone} ) \\ = ( 48 µg / mL \div 64 µg / mL ) + ( 96 µg / mL \div 256 µg / mL ) \\ = 0.75 + 0.375 \\ = 1.125\end{matrix}$$

With a FICI of 1.125, the interaction between thymoquinone and peptide H5 is classified as indifferent (1<FICI<4), although it is very close to the additive threshold (FICI≤1), suggesting a weak indifferent effect that borders on additivity. These findings suggest that while both combinations show beneficial interactions, the thymoquinone-peptide H3 combination exhibits a more favorable interaction profile compared to the thymoquinone-peptide H5 combination.

Time-kill kinetics assays were performed to evaluate the bactericidal activity of thymoquinone and peptides H3 and H5, both individually and in combination, over time. The thymoquinone-peptide H3 combination at their respective sub-MICs (32 μg/mL for thymoquinone and 64 μg/mL for peptide H3) demonstrated enhanced bactericidal activity compared to either compound alone at these concentrations. After 8 hours of exposure, the thymoquinone-peptide H3 combination reduced the bacterial population by approximately 3.2 log 10 CFU/mL, which was significantly greater than the reductions achieved by thymoquinone alone (1.1 log 10 CFU/mL) or peptide H3 alone (0.8 log 10 CFU/mL) at the same concentrations (p<0.001). Complete bacterial eradication was observed after 12 hours of exposure to the combination, while neither compound alone at these concentrations achieved complete killing even after 24 hours. The thymoquinone-peptide H5 combination at their respective sub-MICs (48 μg/mL for thymoquinone and 96 μg/mL for peptide H5) also demonstrated enhanced bactericidal activity compared to either compound alone at these concentrations, although to a lesser extent compared to the thymoquinone-peptide H3 combination. After 8 hours of exposure, the thymoquinone-peptide H5 combination reduced the bacterial population by approximately 2.5 log 10 CFU/mL, which was significantly greater than the reductions achieved by thymoquinone alone (1.4 log 10 CFU/mL) or peptide H5 alone (0.6 log 10 CFU/mL) at the same concentrations (p<0.01). Complete bacterial eradication was observed after 16 hours of exposure to the combination, while neither compound alone at these concentrations achieved complete killing even after 24 hours. These findings indicate that both combinations exhibit enhanced bactericidal effects against MRSA, with the thymoquinone-peptide H3 combination showing more pronounced enhancement compared to the thymoquinone-peptide H5 combination. The enhanced bactericidal activity of the combinations, particularly at sub-MIC concentrations, further supports the synergistic or additive interactions between these compounds.

Molecular docking studies were performed to investigate the potential molecular basis of the synergistic interactions between thymoquinone and peptides H3 and H5. Sequential docking simulations, where thymoquinone was first docked to the target protein followed by docking of the peptide to the same protein with thymoquinone in its docked position, provided insights into potential cooperative binding mechanisms. For the thymoquinone-peptide H3 combination, sequential docking to the bacterial membrane revealed interesting cooperative binding patterns. When thymoquinone was docked first, it appeared to create a hydrophobic anchor point that facilitated the subsequent binding of peptide H3. The binding energy of peptide H3 to the membrane with pre-docked thymoquinone (−10.1 kcal/mol) was lower (more favorable) than its binding energy to the membrane alone (−9.3 kcal/mol), suggesting enhanced binding affinity in the presence of thymoquinone. Furthermore, the presence of thymoquinone appeared to induce conformational changes in the membrane structure that exposed additional binding sites for peptide H3. This cooperative binding mechanism may explain the enhanced membrane-disrupting effects observed for the thymoquinone-peptide H3 combination. Similar cooperative binding patterns were observed for the thymoquinone-peptide H5 combination, although the enhancement in binding affinity was less pronounced. The binding energy of peptide H5 to the membrane with pre-docked thymoquinone (−9.2 kcal/mol) was only slightly lower than its binding energy to the membrane alone (−8.9 kcal/mol), which may explain the weaker synergistic interaction observed for this combination. In addition to cooperative binding to the membrane, molecular docking studies suggested that thymoquinone and the peptides may also interact with different but complementary targets in MRSA. Thymoquinone showed favorable binding to enzymes involved in cell wall biosynthesis, such as penicillin-binding proteins (PBPs), while the peptides exhibited stronger binding to membrane phospholipids and proteins involved in membrane integrity. This multi-target approach, where thymoquinone and the peptides simultaneously attack different but interconnected cellular components, may contribute to their synergistic antimicrobial effects. By disrupting both cell wall biosynthesis and membrane integrity, the combinations may overwhelm the bacterial defense mechanisms, leading to enhanced bacterial killing.

The synergistic interactions between thymoquinone and peptides H3 and H5 have significant implications for addressing the growing problem of antimicrobial resistance, particularly in MRSA. By combining these compounds, it is possible to achieve effective antimicrobial activity at lower concentrations of each compound compared to when they are used individually. This dose reduction may help minimize the selective pressure that drives the development of resistance. Furthermore, the multi-target approach of these combinations, where different but interconnected cellular components are simultaneously attacked, may make it more difficult for bacteria to develop resistance. Bacteria would need to simultaneously evolve multiple resistance mechanisms to overcome the combined effects of these compounds, which is statistically less likely than developing resistance to a single compound with a single target. The natural origin of these compounds may also offer advantages in terms of resistance development. Unlike conventional antibiotics, which often have specific molecular targets that can be easily modified by bacteria to confer resistance, natural compounds like thymoquinone and plant-derived peptides often have multiple modes of action and targets, making resistance development more challenging. Long-term exposure studies would be valuable to assess the potential for resistance development against these combinations. However, based on their mechanisms of action and the synergistic interactions observed, these combinations hold promise as strategies to combat antimicrobial resistance in MRSA.

The synergistic antimicrobial effects of thymoquinone and peptides H3 and H5 against MRSA suggest several potential therapeutic applications. These combinations could be developed into novel antimicrobial formulations for the treatment of MRSA infections, which are often difficult to treat due to resistance to conventional antibiotics. Topical formulations, such as creams, ointments, or wound dressings containing these combinations, could be particularly useful for the treatment of MRSA skin and soft tissue infections. The lipophilic nature of thymoquinone and the enhanced hydrophobicity of the modified peptides may facilitate their penetration into the skin, allowing them to reach the site of infection. Inhalation formulations, such as nebulized solutions or dry powder inhalers, could be developed for the treatment of MRSA respiratory infections. The volatile nature of thymoquinone may be advantageous for respiratory delivery, while the peptides could provide sustained antimicrobial activity at the infection site. For systemic applications, these combinations would need to be formulated to overcome potential pharmacokinetic challenges, such as rapid metabolism or poor bioavailability. Nanoparticle-based delivery systems, liposomal formulations, or other advanced drug delivery technologies could be explored to enhance the stability and bioavailability of these compounds for systemic use. In addition to their direct antimicrobial effects, these combinations may also have potential as adjuvants to conventional antibiotics. By enhancing the permeability of bacterial membranes, they may facilitate the entry of antibiotics into bacterial cells, potentially restoring the effectiveness of antibiotics to which MRSA has developed resistance.

The present invention aims to investigate the antimicrobial activity of thymoquinone and chemically modified peptides (H3 and H5) derived from date palm seeds against methicillin-resistant Staphylococcus aureus (MRSA), with a particular focus on their potential synergistic interactions. The study employed a comprehensive approach, utilizing various microbiological, microscopic, and molecular techniques to elucidate the antimicrobial properties of these compounds and the mechanisms underlying their interactions. Thymoquinone demonstrated significant antimicrobial activity against MRSA with a minimum inhibitory concentration (MIC) of 64 μg/mL. Time-kill kinetics analysis revealed that thymoquinone exhibited concentration-dependent bactericidal activity, achieving complete bacterial eradication at 2×MIC within 12 hours. The chemically modified peptides H3 and H5, both derived from date palm seeds, demonstrated notable antimicrobial activity against MRSA with identical MIC values of 256 μg/mL. Time-kill kinetics analysis showed that both peptides exhibited time-dependent bactericidal activity, although with slower killing rates compared to thymoquinone. The most significant finding of this invention is the synergistic interaction between thymoquinone and peptide H3 against MRSA. When combined, the MIC of thymoquinone decreased from 64 μg/mL to 32 μg/mL, while the MIC of peptide H3 decreased from 256 μg/mL to 64 μg/mL. The fractional inhibitory concentration index (FICI) for this combination was 0.75, indicating a strong additive effect bordering on synergy. Time-kill kinetics analysis demonstrated that the thymoquinone-peptide H3 combination at sub-MIC concentrations exhibited enhanced bactericidal activity compared to either compound alone, achieving complete bacterial eradication within 12 hours. The combination of thymoquinone with peptide H5 also showed beneficial interactions, although to a lesser extent compared to the thymoquinone-peptide H3 combination. The FICI for this combination was 1.125, indicating a weak indifferent effect bordering on additivity. Nevertheless, time-kill kinetics analysis revealed enhanced bactericidal activity for this combination compared to the individual compounds. Molecular docking studies provided insights into the potential molecular basis of these synergistic interactions. Sequential docking simulations suggested cooperative binding mechanisms, where thymoquinone creates a hydrophobic anchor point that facilitates the subsequent binding of the peptides to the bacterial membrane. Furthermore, thymoquinone and the peptides appeared to interact with different but complementary targets in MRSA, potentially leading to a multi-target approach that overwhelms bacterial defense mechanisms. These findings have significant implications for addressing the growing problem of antimicrobial resistance, particularly in MRSA. The synergistic combinations of thymoquinone and peptides H3 and H5 offer the potential for effective antimicrobial activity at lower concentrations of each compound, which may help minimize the selective pressure driving resistance development. The multi-target approach of these combinations may also make it more difficult for bacteria to develop resistance, as they would need to simultaneously evolve multiple resistance mechanisms.

The significance of the present invention lies in its contribution for novel antimicrobial strategies to combat the growing threat of antimicrobial resistance, particularly in MRSA, wherein the findings of this invention offer several important contributions to this field. Firstly, this invention aims to expand the understanding of the antimicrobial properties of natural compounds derived from plants. By elucidating the antimicrobial mechanisms of thymoquinone and date palm-derived peptides against MRSA, the invention highlights the potential of natural products as sources of novel antimicrobial agents. Secondly, this invention demonstrates the value of chemical modifications in enhancing the antimicrobial properties of naturally occurring peptides. The acetylation of lysine residues in peptides H3 and H5 increased their hydrophobicity, potentially improving their interaction with bacterial membranes. This approach of modifying natural peptides to enhance their antimicrobial activity represents a promising strategy for developing new antimicrobial agents with improved efficacy and stability. Thirdly, and perhaps most significantly, this invention highlights the potential of combination therapy as a strategy to combat antimicrobial resistance. The synergistic interaction between thymoquinone and peptide H3, and to a lesser extent between thymoquinone and peptide H5, demonstrates that combining compounds with different but complementary mechanisms of action can lead to enhanced antimicrobial effects. This approach not only allows for the use of lower concentrations of each compound, potentially reducing side effects, but also may delay the development of resistance by requiring bacteria to simultaneously evolve multiple resistance mechanisms. Furthermore, this invention contributes to the understanding of the molecular mechanisms underlying synergistic antimicrobial interactions. The findings suggest that enhanced membrane disruption and cooperative binding to multiple targets are key factors in the synergistic effects observed. This mechanistic insight may guide the rational design of other synergistic combinations targeting MRSA and other resistant pathogens.

In an implementation, antimicrobial formulations for the treatment of MRSA infections is developed, wherein the formulation includes the combinations of thymoquinone and peptides H3 and H5, and is used for skin and soft tissue infections, inhalation formulations for respiratory infections, or systemic formulations with appropriate delivery systems. Additionally, the prepared formulations aim to restore the effectiveness of antibiotics to which MRSA has developed resistance.

In an embodiment, the invention has several limitations that should be addressed. One significant limitation is the focus on a single MRSA strain (ATCC 43300). The antimicrobial activity and synergistic interactions observed in the invention may not be generalizable to all MRSA strains, particularly clinical isolates with different resistance profiles. Therefore there is a need for evaluating these combinations against a broader range of MRSA strains, including clinical isolates from various sources and with different resistance patterns. Another limitation is the in vitro nature. While in vitro experiments provide valuable insights into antimicrobial activity and mechanisms of action, they may not fully predict in vivo efficacy. Factors such as protein binding, tissue distribution, metabolism, and host immune responses can significantly influence the effectiveness of antimicrobial agents in vivo. Animal models of MRSA infection would be necessary to validate the efficacy of these combinations under more physiologically relevant conditions. The invention also did not comprehensively assess the potential toxicity of thymoquinone and peptides H3 and H5 to human cells. While thymoquinone has been reported to have a favorable safety profile in various studies, its effects on human cells at the concentrations required for antimicrobial activity need to be thoroughly evaluated. Similarly, the safety profile of the chemically modified peptides H3 and H5 needs to be established, particularly considering their membrane-disrupting properties, which may not be selective for bacterial membranes. The stability of these compounds under physiological conditions was not extensively investigated in this invention. Thymoquinone is known to be susceptible to oxidation and degradation, which may limit its shelf life and in vivo efficacy. The peptides may be susceptible to enzymatic degradation by proteases present in biological fluids, which could limit their bioavailability and duration of action. Stability studies under various conditions would be necessary to address these concerns. The molecular docking studies, while providing valuable insights into potential binding mechanisms, have inherent limitations. These computational simulations are based on theoretical models and may not fully capture the complexity of molecular interactions in a biological system. Experimental validation of the predicted binding interactions using techniques such as isothermal titration calorimetry or surface plasmon resonance would strengthen these findings. Additionally, the invention did not explore the potential for resistance development against these combinations over extended periods. While the multi-target approach of these combinations may theoretically make resistance development more difficult, experimental evidence from long-term exposure studies would be necessary to confirm this hypothesis. Finally, the invention did not address the practical aspects of formulating these compounds into stable, effective, and user-friendly pharmaceutical products. The hydrophobic nature of thymoquinone and the amphipathic nature of the peptides may present challenges for formulation and delivery, which would need to be addressed in future studies. Based on the findings and limitations, several directions for future research can be identified to further advance the understanding of the synergistic antimicrobial effects of thymoquinone and date palm-derived peptides against MRSA and to translate these findings into practical applications.

Expanding the scope of antimicrobial testing to include a diverse panel of MRSA strains, including clinical isolates with various resistance profiles, would provide a more comprehensive assessment of the potential clinical utility of these combinations. Additionally, testing against other clinically relevant pathogens, including other resistant Gram-positive bacteria, Gram-negative bacteria, and fungi, would help determine the spectrum of activity of these combinations. In vivo studies using appropriate animal models of MRSA infection (e.g., skin and soft tissue infection, pneumonia, or systemic infection models) would be essential to validate the efficacy of these combinations under physiologically relevant conditions, wherein various dosing regimens, routes of administration, and treatment durations needs to be evaluated, to optimize the therapeutic potential of these combinations. Comprehensive toxicity studies are necessary to establish the safety profile of thymoquinone and peptides H3 and H5, both individually and in combination, wherein in vitro cytotoxicity assays using relevant human cell lines, hemolysis assays to assess potential damage to red blood cells, and in vivo toxicity studies in animal models should be included to evaluate systemic effects and organ-specific toxicity. Pharmacokinetic and pharmacodynamic studies would provide valuable information about the absorption, distribution, metabolism, excretion, and bioavailability of these compounds in vivo, which would help determine appropriate dosing regimens and identify potential limitations in bioavailability that may need to be addressed through formulation strategies. Stability studies under various conditions (e.g., different temperatures, pH values, and in the presence of biological fluids) would help assess the shelf life and in vivo stability of these compounds. Strategies to enhance stability, such as chemical modifications, encapsulation, or protective formulations, could be explored based on these findings. More detailed mechanistic studies using advanced techniques such as transcriptomics, proteomics, and metabolomics would provide deeper insights into the molecular mechanisms underlying the synergistic interactions between thymoquinone and the peptides. These studies could identify specific cellular pathways and targets affected by these combinations, potentially revealing additional mechanisms beyond membrane disruption. Long-term exposure studies would be valuable to assess the potential for resistance development against these combinations. These studies should involve serial passage of MRSA in the presence of sub-inhibitory concentrations of the combinations over extended periods, followed by susceptibility testing and whole-genome sequencing to identify potential resistance mechanisms. Formulation development studies should explore various strategies to overcome the challenges associated with the physicochemical properties of these compounds. For topical applications, formulations such as creams, ointments, or wound dressings could be developed and optimized for stability, release kinetics, and skin penetration. For potential systemic applications, advanced delivery systems such as nanoparticles, liposomes, or other carrier systems could be investigated to enhance bioavailability and target delivery. Combination studies with conventional antibiotics would be interesting to explore potential synergistic or additive interactions that could restore the effectiveness of antibiotics to which MRSA has developed resistance. These studies could identify promising triple combinations (thymoquinone+peptide+antibiotic) with enhanced efficacy against resistant strains. Finally, scaling up the production of these compounds, particularly the peptides, and optimizing their extraction or synthesis processes would be necessary for practical applications. Cost-effective and environmentally friendly methods for large-scale production would be essential for the commercial viability of these combinations as antimicrobial agents. In conclusion, this research has demonstrated the promising synergistic antimicrobial effects of thymoquinone and chemically modified peptides derived from date palm seeds against MRSA. These findings contribute to our understanding of natural antimicrobial compounds and highlight the potential of combination therapy as a strategy to combat antimicrobial resistance. While several limitations and challenges need to be addressed, the results of this study provide a solid foundation for future research aimed at developing novel antimicrobial strategies based on these natural compounds. With continued research and development, these combinations may eventually contribute to our arsenal of weapons against the growing threat of antimicrobial resistance.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A process for preparation of a chemically-modified peptide derived from seed material, comprising the steps of:
mechanically reducing mature seed structures into a particulate fraction having a controlled particle size distribution not exceeding 500 micrometers;
subjecting the particulate fraction to continuous solvent-driven lipid displacement using a non-polar extraction medium under refluxing conditions until residual lipid content in the extracted solids is reduced below 2% w/w;
introducing the defatted substrate into an aqueous buffered medium adjusted to a pH between 7.0 and 7.6 at a mass-to-volume ratio between 1:5 and 1:15, followed by low-temperature agitation at a temperature not exceeding 10 °C for at least four hours to induce solubilization of intrinsic peptide-bearing proteins without promoting thermally induced aggregation;
separating insoluble plant-derived fibers from the solubilized protein phase by centrifugal clarification at a relative centrifugal force of at least 8,000×g;
introducing into the clarified supernatant a precipitation-inducing agent at a level sufficient to selectively aggregate macromolecular proteins while permitting peptide fragments shorter than fifteen residues to remain in solution, thereby producing a peptide-enriched supernatant;
feeding the peptide-enriched supernatant through a first chromatographic separation stage operating under hydrodynamic-radius-dependent resolution conditions to remove species exceeding 2 kDa, followed by a second chromatographic separation stage employing a progressively increasing organic-phase gradient to elute fractions in accordance with surface hydrophobicity, thereby isolating an eluate containing a polypeptide consisting of eleven residues and comprising an internal cysteine-containing motif;
introducing a terminal-modifying reagent into the isolated eluate in a molar ratio of at least fifty-fold relative to the peptide under alkaline-buffered conditions maintained between pH 7.8 and 8.4, thereby causing covalent modification at one or both termini of the peptide;
terminating the modification reaction through controlled introduction of a quenching species that neutralizes unreacted modifying reagent, followed by isolating the modified peptide using an organic-phase gradient elution configured to separate chemically modified peptide species from any residual unmodified species; and
drying the resulting purified peptide under reduced-pressure dehydration conditions, wherein the non-polar extraction medium comprises analytical-grade n-hexane having a purity of at least 99%, introduced in a solvent-to-solid ratio of 10:1 v/w, circulated continuously at reflux temperature for a minimum of six hours, and thereafter subjected to incremental solvent replacement at intervals of 60 minutes until the outgoing solvent stream exhibits a steady-state absorbance below 0.02 AU at 280 nm, indicative of maximal extraction of lipid components inherent to Phoenix dactylifera seed matrices, wherein the aqueous buffered medium used to disperse the defatted Phoenix dactylifera seed substrate comprises a phosphate-based buffer prepared at an ionic strength between 50 and 80 mM, supplemented with 0.1-0.5% w/v sodium chloride to stabilize solubilized protein structures, and wherein the hydration step includes staged addition of the buffer to prevent local oversaturation zones, thereby promoting homogeneous solubilization of polypeptide-bearing macromolecules, and wherein the low-temperature agitation of the buffered dispersion is performed using a horizontal orbital shaker calibrated to operate at 180-220 oscillations per minute, with the vessel partially submerged in a chilled circulating bath maintained at 6-9 °C, ensuring suppression of auto-oxidative reactions associated with cysteine-containing peptide motifs found in Phoenix dactylifera seed protein fractions, and wherein the centrifugal clarification step employs a centrifugation vessel fabricated from inert polymeric material to avoid adsorption of short-chain peptides, and wherein the vessel is filled to no more than 70% of its nominal volume to maintain consistent sedimentation rates, with the clarified supernatant immediately transferred into a nitrogen-flushed storage container to minimize oxidative modification of susceptible amino-acid side chains.

2. The process of claim 1, wherein the seed material comprises seeds of Phoenix dactylifera that have been manually separated from pericarp residues, washed with deionized water to remove adhering soil particulates, air-dried at temperatures not exceeding 35 °C to prevent thermal denaturation of endogenous proteins, and subsequently conditioned to a uniform moisture content between 10% and 12% prior to mechanical milling, such conditioning ensuring consistent fragmentation efficiency and preventing differential stress-induced generation of undesirable ultrafine particulates during milling, and wherein the mechanical reduction of the Phoenix dactylifera seeds is carried out using a rotational impact mill equipped with high-inertia stainless-steel blades, operated at a rotational speed between 8,000 and 12,000 RPM while the milling chamber is externally cooled using a recirculating coolant maintained below 15 °C, thereby constraining frictional heat generation and preserving functional peptide conformations that would otherwise degrade under localized thermal hotspots.

3. The process of claim 1, wherein the precipitation-inducing agent comprises analytical-grade ammonium sulfate having a certified assay purity above 98%, added gradually over a period of 20-40 minutes while maintaining agitation to prevent localized supersaturation and protein denaturation, such that proteins above 10 kDa selectively aggregate while polypeptides shorter than fifteen residues derived from Phoenix dactylifera remain fully solubilized in the supernatant.

4. The process of claim 1, wherein the size-based chromatographic separation is performed using a column packed with cross-linked polymeric beads having a pore distribution centered around 1.2 nm to selectively exclude species larger than 2 kDa, and wherein the eluent is continuously monitored for refractive index deviation to ensure precise discrimination between peptide-enriched and protein-contaminated fractions.

5. The process of claim 1, wherein the hydrophobic-interaction separation employs an organic solvent gradient comprising water as the aqueous phase and acetonitrile as the organic phase, both containing 0.05-0.1% acidic modifier to maintain peptide ionization stability, the gradient being raised in a controlled linear profile of no more than 1% organic solvent increase per minute to achieve temporally resolved elution of the eleven-residue Phoenix dactylifera peptide fraction.

6. The process of claim 1, wherein the isolated eluate containing the eleven-residue peptide is maintained at a temperature between 2 °C and 6 °C prior to chemical modification, and wherein dissolved oxygen levels within the eluate are reduced to below 2 mg/L by sparging with inert gas to inhibit oxidation of internal cysteine-bearing motifs derived from Phoenix dactylifera.

7. The process of claim 1, wherein the terminal-modifying reagent comprises analytical-grade acetic anhydride for N-terminal acetylation, benzoyl chloride for aromatic acylation, or an activated N-hydroxysuccinimide derivative for alternative terminal capping, each reagent being freshly prepared immediately before addition to minimize hydrolysis and ensure maximized coupling efficiency, and wherein the terminal-modifying reagent is added in a stepwise fashion over a period of 10-15 minutes to avoid sudden pH excursions, and wherein the pH is continuously monitored using a corrosion-resistant probe and adjusted using dilute alkali to remain strictly within the range of 7.8-8.4 throughout the derivatization reaction.

8. The process of claim 1, wherein the quenching species comprises a dilute aqueous primary amine or hydroxide solution added in a controlled rate not exceeding 0.5 mL/min to neutralize unreacted modifying reagent while avoiding localized alkaline hotspots that may induce peptide backbone cleavage, and wherein post-modification purification is performed using a gradient elution system wherein the organic phase is increased in a shallow, multi-segment profile designed to differentiate mono-modified from di-modified peptide species, thereby ensuring isolation of a homogeneously modified peptide fraction originating from Phoenix dactylifera.

9. The process of claim 1, wherein the purified peptide solution is concentrated under reduced pressure using a vacuum level between 120 and 200 mbar, while the vessel is immersed in a temperature-regulated bath set between 20 °C and 25 °C to prevent thermally induced racemization of amino-acid residues, and wherein the final drying step employs staged dehydration beginning with partial solvent removal under gentle vacuum, followed by a terminal dehydration phase conducted at pressures below 30 mbar until the resulting peptide achieves a moisture content below 5% w/w and exhibits stable flow behavior suitable for subsequent formulation.

10. The process of claim 1, wherein storage of intermediate fractions is carried out in amber-tinted containers composed of non-adsorptive polymeric material to prevent photodegradation or peptide loss through surface binding, each container being sealed with low-oxygen-permeability closures to maintain peptide integrity throughout the multi-stage preparation process, and wherein the final purified, modified peptide is collected in a contaminant-free receiving vessel pre-conditioned with a small aliquot of the same organic solvent used in final elution to minimize adsorption, and subsequently transferred to a sealed, desiccated storage environment maintained below 25% relative humidity to ensure long-term chemical stability of the chemically-modified Phoenix dactylifera peptide.

11. The process of claim 1, wherein the preparation of the aqueous buffered medium used for solubilizing the defatted Phoenix dactylifera seed-derived substrate comprises dissolving a predetermined quantity of disodium hydrogen phosphate and monosodium dihydrogen phosphate in deionized water to obtain a total phosphate concentration between 45 mM and 85 mM, subsequently adjusting conductivity through the incremental addition of analytical-grade sodium chloride to achieve an ionic strength of 4.5-6.0 mS/cm, and pre-filtering the buffer through a low-protein-binding membrane having a pore size of 0.22 micrometers to remove particulate contaminants prior to hydration of the protein-bearing substrate, such that buffer homogeneity and physicochemical stability are maintained throughout the multi-hour low-temperature solubilization stage, thereby preventing premature precipitation of small-mass, cysteine-containing peptide fragments inherent in the Phoenix dactylifera matrix.

12. The process of claim 1, wherein the peptide-enriched supernatant resulting from selective precipitation of high-molecular-weight proteins is subjected to a controlled dilution step using chilled deionized water equilibrated to 4-6 °C, the dilution being performed gradually at a rate not exceeding 5% volumetric increase per minute to reduce solution viscosity and optimize mass-transfer properties during subsequent chromatographic loading, and wherein the diluted supernatant is maintained under constant gentle agitation using a magnetically driven stir system to prevent localized concentration gradients, thereby ensuring uniform interaction of Phoenix dactylifera-derived short-chain polypeptides with the bead matrix during size-selective chromatographic processing.

13. The process of claim 1, wherein the first chromatographic stage is performed using a column packed with cross-linked polysaccharide beads possessing a calibrated swelling index between 130% and 160%, said beads being pre-equilibrated with the same aqueous buffer used during initial substrate solubilization to minimize osmotic shock, and wherein the flow rate through the column is maintained between 0.4 and 0.7 column volumes per hour to prevent displacement of smaller Phoenix dactylifera peptide species into the excluded volume region, such slow, controlled throughput enabling accurate isolation of the eleven-residue fraction while minimizing co-elution of undesirable oligopeptide contaminants.

14. The process of claim 1, wherein the reaction environment for terminal-end chemical modification is established by pre-conditioning the reaction vessel with a dilute alkaline rinse solution to neutralize residual acidic species, followed by introducing the isolated eluate containing the eleven-residue Phoenix dactylifera peptide and equilibrating the solution temperature to 22-25 °C with a tolerance of ±0.2 °C, and wherein the modifying reagent is incrementally introduced using a calibrated syringe pump capable of maintaining a dosage variation not exceeding 1%, thereby avoiding abrupt concentration spikes and supporting uniform reagent-peptide contact across the entire reaction volume, resulting in consistent modification along each peptide molecule present.

15. The process of claim 1, wherein post-reaction neutralization is facilitated by the controlled addition of a quenching agent comprising a dilute aqueous ammonia solution adjusted to pH 9.0-9.3, dispensed using a micro-controlled addition system that continuously monitors solution pH in real time, adjusting addition rate to maintain pH fluctuations within ±0.05 units, thereby ensuring that excess modifying reagent is fully deactivated without inducing structural rearrangements or partial hydrolysis of the chemically-modified Phoenix dactylifera peptide backbone.

16. The process of claim 1, wherein the final dehydration stage is executed in a two-phase protocol comprising an initial solvent-removal phase conducted under reduced pressure between 80 and 150 mbar with constant rotary agitation to uniformly distribute thermal input, followed by a secondary deep-drying phase initiated only after the residual solvent content has decreased below 10% w/v, the second phase being conducted at pressures below 25 mbar and temperatures not exceeding 28 °C, with continuous moisture monitoring via an integrated capacitive hygrometry sensor, thereby ensuring that the final chemically-modified Phoenix dactylifera peptide exhibits a stable, low-moisture, free-flowing powder consistency suitable for long-term storage and downstream incorporation into formulation systems.
